SEQUENCE LISTING (25 sequences)

SEQ ID NO: 1 - DNA, 1695 bp, Influenza A virus
  source 1..1695: mol_type = other DNA
atgaaagctaaactactaatcctgttgtgtgcactttcaactacagatgcagacacaatatgtataggataccatgctaacaattcaaccgacaccgttgacacagtcctagaaaagaatgtgacagtaacacactctgtcaacctccttgaggacagtcacaatgggaaactatgtaaactgaagggagtggccccactgcaattgggaaaatgcagcatcgcaggatggatactagggaacccagagtgtgaatcactgctttctaagaaatcatggtcctatattgcagaagcaccaaatgctgagaatggaatatgttacccagggcacttctccgactacgaagagctgagggagcaattaagttcagtatcttcattcgagaggttcgaaatatttcctaaggagagttcatggcccaaacacagcataggagcaactgcatcatgctccaaacaagggaaaagcagtttttatacaaatctgctatggctaactgaaaaaaatgggtcttatccaaatttgaccatgtcctatatgaacgacaaagagagagaagtccttgtgctatggggagtccatcatccgtccaacatagaggaccaaagagcaatctatcgcaaagaaactgcttatgtttctgtaatgtcatcacactacaacaagaggttcaccccagagattgcaaaaagacccaaagtaaggaatcaagaggggagaatcaactactactggacactgctggaacccagggacacaataatatttgaagcaaatgggaatttgatagcaccatggtatgcttttgcattgaatagaggctttgagtcaggaatcattgtctcaaatgcatcgatggatgagtgtaacgcgaagtgtcaaacgccccgaggagcgataaacaatagcctgcctttccagaatgtacacccaatcacaataggggaatgccccaagtatgtaaagagtacaaaattaaagatggctacaggacttagaaacattccatccatccaatccagaggtttgtttggagccattgctggtttcattgaagggggatggaccggaatgatagatggatggtatggttatcatcatcagaatgaacagggttctggctatgctgcagatcaaaaaagcacacaaaatgccattaatgggattacaaacaaggtgaactctgttatcgataaaatgaacactcaatttacagctgtaggtaaagaattcaacagattagaaaaaagaatggaaaacctgaataagaaggttgatgacggttttcttgacatttggacgtataatgcagaattgttggttctgctggagaatgagaggactttggattttcatgattcaaatgtgaagagcttatatgaaggagtaaaggcccaattgaagaataatgccaaagaagtaggaaacgggtgttttgaattctaccacaagtgtgacaacgaatgcatggacagtgtaaaaaatggaacttatgactatccaagatactcagaggaatcaaaattaaacagagaacaaattgatggagtggaattgaaatcaatgggagtctatcagatcctggcgatatactcaactgccgccagttcattggtgcttttagtcaccctgggggcaatcagtttttggatgtgttctaatggttctttgcagtgcagaatatgtatctga

SEQ ID NO: 2 - DNA, 1695 bp, synthetic construct
  misc_feature 1..1695: note = >EUH1-17 Swine optimized sequence
  source 1..1695: mol_type = other DNA
atgaaagccaagctcctcattctcctttgtgccctgtccacgaccgacgctgatactatctgcatcggctaccatgccaacaactccactgataccgtggacaccgtgctggaaaagaacgtgaccgtgactcactcggtgaacctccttgaggacagccacaacgggaagctctgcaagctgaagggcgtggcacccctacaactgggaaagtgttccatcgcgggctggattctgggaaaccccgagtgcgaatcccttctgtcgaaaaagtcatggtcctacattgccgaggccccgaatgctgagaacggcatttgctacccgggacacttcagcgattacgaagaactgcgggaacagctgtcgagcgtgtcaagtttcgagaggttcgagatcttcccgaaggaaagctcatggccgaagcacagcatcggcgccaccgcctcgtgttcgaaacagggaaagtcctcgttctacactaacctcctgtggctgaccgagaagaatgggtcctatcctaacctgaccatgtcgtacatgaacgacaaggaacgggaagtgctcgtgttgtggggggtgcaccatccatcaaatatcgaggaccagagggccatctaccgcaaagaaactgcctacgtgtccgtgatgtctagccactacaacaagcgcttcacgcctgaaatcgccaaaagacccaaggtccgcaaccaagagggcagaatcaattactactggaccctcctggagccccgggacaccataatctttgaagcaaatggaaacctcattgcgccttggtacgccttcgccctgaaccggggtttcgagtccggaattatcgtcagcaacgcctccatggacgagtgtaacgcgaagtgccagaccccgcgcggcgctatcaacaacagcctgcctttccaaaacgtgcacccaatcaccatcggcgaatgccccaaatacgtgaagtccactaagctgaagatggcaacaggactgcggaacatcccgtcaattcagtcccgagggttgttcggggcaatcgccgggttcattgagggcggatggaccggaatgattgacggttggtacggataccaccaccagaacgaacagggttccggttatgcggcggatcagaagtccacccagaacgccatcaacggcatcaccaacaaggtcaactccgtcatcgacaagatgaacacccagttcactgccgtgggaaaggagttcaaccggctggagaagcggatggaaaatctgaacaagaaagtcgacgacggcttcctggacatttggacctacaacgccgaactgctggtgctgctggaaaacgaaagaactctcgactttcatgattcgaacgtgaagtcactgtacgaaggagtgaaggcccagctcaagaacaacgccaaggaagtcggaaacggttgcttcgagttttaccataagtgcgacaacgagtgcatggatagcgtgaagaacggaacctacgactacccccggtattccgaagagtcgaagctcaacagagaacagatcgatggcgtggagctgaagtccatgggagtgtaccagatcctggcgatctactccactgcggctagctccctggtgctgttggtcaccttgggggctatctctttctggatgtgctccaacggttccctgcaatgccgcatttgcatttga

SEQ ID NO: 3 - protein, 564 aa, Influenza A virus
  REGION 1..564: note = >ALX30160.1 hemagglutinin [Influenza A virus (A/swine/Italy/3033-1/2015(H1N2))]
MKAKLLILLCALSTTDADTICIGYHANNSTDTVDTVLEKNVTVTHSVNLLEDSHNGKLCKLKGVAPLQLGKCSIAGWILGNPECESLLSKKSWSYIAEAPNAENGICYPGHFSDYEELREQLSSVSSFERFEIFPKESSWPKHSIGATASCSKQGKSSFYTNLLWLTEKNGSYPNLTMSYMNDKEREVLVLWGVHHPSNIEDQRAIYRKETAYVSVMSSHYNKRFTPEIAKRPKVRNQEGRINYYWTLLEPRDTIIFEANGNLIAPWYAFALNRGFESGIIVSNASMDECNAKCQTPRGAINNSLPFQNVHPITIGECPKYVKSTKLKMATGLRNIPSIQSRGLFGAIAGFIEGGWTGMIDGWYGYHHQNEQGSGYAADQKSTQNAINGITNKVNSVIDKMNTQFTAVGKEFNRLEKRMENLNKKVDDGFLDIWTYNAELLVLLENERTLDFHDSNVKSLYEGVKAQLKNNAKEVGNGCFEFYHKCDNECMDSVKNGTYDYPRYSEESKLNREQIDGVELKSMGVYQILAIYSTAASSLVLLVTLGAISFWMCSNGSLQCRICI

SEQ ID NO: 4 - DNA, 1701 bp, Influenza A virus
  misc_feature 1..1701
  source 1..1701: mol_type = other DNA
atgaaagcaaaattatttgtattattctgtgcatttactgcactgaaagctgacaccatttgtgtaggctatcatgctaacaattccacagacactgtcgacacgatactggaaaagaatgttactgttacccactcagttaatttactagaaaacagccacaatggaaaactctgcagcctgaatggaaaggctccgttgcaactggagaactgcaacgtagcaggatggatccttggtaatccagaatgtgactttctgctcacagcgaattcatggtcctacataatagagacttcaaattcaaaaaatggaacatgctatcctggagaattcactgattatgaggaactaagggagcaactgagtacagtttcttcatttgaaagatttgaaattttcccaaaagcaacctcatggccaaatcatgagacaaccaaaggtaccacagttgcatgctcccactctggagccaacagtttttatcggaacttgctatggatagtaaagaaaggaaactcctatcccaagctcagcaagtcatacacaaacaacaaagggaaagaagtgcttgtaatctggggagtgcatcacccccctacagacagtgaccaacaaaccctctatcagaataatcacacatatgtttcagttggatcatcaaaatactaccaaaggttcacaccagaaatagtagccagacctaaagtcagagagcaagcgggcagaatgaattattattggatactattagatcaaggagacaccataacatttgaagccacggggaatttaatagcaccatggcatgcatttgcattgaataagggctctagctctggaattatgatgtcagatgctcatgttcaaaattgcactacaaagtgccaaactcctcatggggcattgaaaagcaatcttccttttcaaaatgtacatcccatcactattggagagtgccccaaatatgttaaaagcacccaactaagaatggcaacaggattaaggaacataccatctatccaatccagaggactttttggggcaattgccggatttattgaaggaggatggacaggaatgatagatggatggtatggatatcaccatcaaaatgagcaaggatctggttatgcagcagataagaaaagcacacaaattgcaattgatgagatcagcaacaaagtaaactcagtaattgagaaaatgaacattcaattcacttcagtgggcaaggagttcaataatttagagaaaaggattgagaatttgaataaaaaggtcgatgatggatttttggatgtgtggacatataacgctgagttactcattttgctcgagaacgaaagaaccctagatttccatgactttaacgtaagaaatttgtatgaaaaggtcaaatcacaactgagaaacaatgccaaggaaatcggtaatggttgttttgagttctatcacagatgtgacaatgaatgcatggaaagtgtaaagaatggcacatataattatcccagatattcagaagaatccaaattaaatagagagaaaatagacggtgtgaaactagaatcattaggagtacatcagattttggcaatctactccacagtcgccagttccctggtattgctagtctccctgggggcaatcagtttctggatgtgctctaatggatcattgcaatgcagaatatgcatttga

SEQ ID NO: 5 - DNA, 1701 bp, synthetic construct
  misc_feature 1..1701: note = >EUH1-3 Swine optimized sequence
  source 1..1701: mol_type = other DNA
atgaaagccaagctgtttgtccttttctgcgccttcaccgcccttaaagccgacaccatctgtgtgggctaccatgctaacaactccaccgatacggtcgacactattctggagaagaacgtgaccgtgacccactccgtgaatctgctggagaatagccacaacggaaagctgtgcagcctgaacggaaaggccccgctgcaactggagaattgtaacgtggccggatggatcctgggcaaccctgaatgcgacttcctgctgaccgccaattcctggtcctacatcatcgagacttccaactccaagaacggaacgtgctaccccggggagttcactgattacgaagaactgcgcgaacagctgtcaactgtgtcctcgttcgagcggttcgaaatcttcccgaaagccacctcgtggcccaatcatgaaaccaccaagggaaccactgtggcctgctcccactctggggcgaactcattctatcgcaacttgctgtggatcgtcaagaagggaaacagttaccctaagctgtccaagtcctacactaacaacaagggaaaggaagtcctcgtgatctggggcgtgcatcacccacccaccgactccgatcagcaaacactgtaccagaacaaccacacctacgtgtccgtgggatcatccaagtactaccagagattcaccccggaaatcgtcgcgcggcccaaggtccgcgaacaagccggccggatgaactactactggatcctgctggaccagggcgacactatcactttcgaggcgaccgggaacctcatcgccccttggcacgcattcgcactgaacaagggatcgagctccgggattatgatgagcgatgctcatgtgcaaaactgtactaccaagtgccagacccctcacggtgccctgaagtccaatttgcccttccaaaatgtgcaccccattaccattggcgaatgcccgaaatacgtgaagtcgacccaactccggatggccaccggccttcggaacatcccatcgatacagtcgaggggtcttttcggagcgattgcgggattcatcgagggcggctggaccggaatgattgacggttggtacggataccaccaccagaacgaacagggctccggatacgccgccgacaaaaagtcgactcagatcgcaattgacgagattagcaacaaggtcaactccgtgatcgagaagatgaatatccagtttactagcgtgggaaaggagttcaacaacctggagaagaggatcgagaacctgaacaagaaagtggacgatggattcctcgacgtgtggacatacaacgccgagctcctcattttgctggagaacgaacggaccctcgacttccatgacttcaacgtgcgcaacctgtacgaaaaggtcaagagccagctgagaaacaacgccaaggaaattggcaacggttgctttgagttttaccaccgctgcgacaacgaatgcatggagagcgtgaagaacgggacctacaactatccgagatactcagaggaatcaaagctgaaccgcgaaaagatcgatggcgtgaagctcgaatcactcggcgtgcaccagatcctggccatctattccactgtggctagctccctcgtgctgctggtgtccctgggtgctatttccttctggatgtgcagcaacgggtcgctccagtgccggatctgtatctga

SEQ ID NO: 6 - protein, 566 aa, Influenza A virus
  REGION 1..566: note = >AKJ81667.1 hemagglutinin [Influenza A virus (A/swine/Italy/28762-3/2013(H1N1))]
MKAKLFVLFCAFTALKADTICVGYHANNSTDTVDTILEKNVTVTHSVNLLENSHNGKLCSLNGKAPLQLENCNVAGWILGNPECDFLLTANSWSYIIETSNSKNGTCYPGEFTDYEELREQLSTVSSFERFEIFPKATSWPNHETTKGTTVACSHSGANSFYRNLLWIVKKGNSYPKLSKSYTNNKGKEVLVIWGVHHPPTDSDQQTLYQNNHTYVSVGSSKYYQRFTPEIVARPKVREQAGRMNYYWILLDQGDTITFEATGNLIAPWHAFALNKGSSSGIMMSDAHVQNCTTKCQTPHGALKSNLPFQNVHPITIGECPKYVKSTQLRMATGLRNIPSIQSRGLFGAIAGFIEGGWTGMIDGWYGYHHQNEQGSGYAADKKSTQIAIDEISNKVNSVIEKMNIQFTSVGKEFNNLEKRIENLNKKVDDGFLDVWTYNAELLILLENERTLDFHDFNVRNLYEKVKSQLRNNAKEIGNGCFEFYHRCDNECMESVKNGTYNYPRYSEESKLNREKIDGVKLESLGVHQILAIYSTVASSLVLLVSLGAISFWMCSNGSLQCRICI

SEQ ID NO: 7 - DNA, 1701 bp, Influenza A virus
  source 1..1701: mol_type = other DNA
atgaagactgtcattgctttaagctacgtcttttgcctggtttttggccaagattttccaggaaagggcaacaacacagcaacactgtgcctagggcatcatgcagtgccaaatgggacattagtgaaaacaatcacagatgatcaaatcgaagtaaccaatgccacagaactggtccaaaatttctcaatgggtaaaatatgcaagaatcctcatcgaattcttgatggggcaaattgtacattggtagactctctattgggggaccctcactgtgatggttttcaaaatgagaaatgggacctcttcattgaacgcagcagggctttcagcaactgctacccttatgatgtgccggagtatacctctcttaggtcactaattgcctcatcaggcaccttggaatttaccaatgaaaatttcaattggactggagttacccaaaatgggggaagcagtgcttgcaagagggggcccaacaacagctttttcagtagattgaactggttgtacaaatcaggaaacacatatccgatgctaaatgttactatgccaaacagtgatgatttcgacaaattgtacatttggggagttcatcatccgagcaccgatagagaacagaccaacctgtacattcaagcatcagggaaaataatagtatccacaaagagaagtcaacaaaccataatcccgaatatcgggtctagaccctgggtaaggggtttatctagtaggataagcatctactggacaatagttaaaccaggagacatcctgataatcaacagcaatggaaacctaattgctcctcggggttacttcaagatacagactgggaaaagttcagtaatgaagtcagatgcacctattggcacctgcaattcagaatgcattactccaaacggaagcatacccaatgataaaccctttcaaaacgtaaacaggatcacatatggggcatgccctcattacatcaagcaaaacactctgaagctagcaacaggaatgcggaatataccagaaagacaaaccagaggtatattcggtgcaatagcagggttcatagagaatggttgggaaggaatggtgaacggttggtacggtttcagacaccaaaattctgagggcataggacaggcagcagatcttaaaagtacccaagcagctatcaaccaaatcaatggaaaactgaatagagtaatcgagaaaacaaacgagaaattccatcaaatcgaaaaggagttctcagaagtagaaggaaggatccaagaccttgaaagatatgttgaagatactaagatagatctttggtcttacaacgcggaacttcttgtcgcactagagaatcaacacacaattgacctgactgactcagaaatgaacaaactctttgaaaaaacaaggaaacaactaagggaaaatgctgaggacatgggaaatggttgcttcaaaatataccacaaatgtgacaactcttgcatggagtcaatcagaaatgggacttacgaccataatgaatacagagacgaagcagtaaacaatcgattccaaatcaaaagtgttgagctgaagtcaggatacaaagactggatcctgtggatttcctttgccatatcatgctttttgctttgcgctatttggatgggattcgtaatatgggcctgccagaaaggcaacattagatgcaatatttgcatctga

SEQ ID NO: 8 - DNA, 1701 bp, synthetic construct
  misc_feature 1..1701: note = >EUH3-4_Swine optimized sequence
  source 1..1701: mol_type = other DNA
atgaaaaccgtcattgcgttgagctatgtgttttgtctcgtgtttggacaagacttcccgggaaagggcaacaacaccgccaccctgtgcctggggcaccatgccgtgcccaacggaactctcgtcaagaccatcaccgacgaccagatcgaggtcaccaatgcgaccgagttggtgcagaatttctccatgggaaagatctgcaagaacccgcaccggattttggatggagccaactgcacactggtggactccctgctgggtgatcctcattgcgacggattccaaaacgagaagtgggatctgttcatcgagagatcccgggccttcagcaactgctacccgtacgacgtgccagagtacactagcttgcggtccctgatcgcctccagcggtaccctggagttcaccaacgagaacttcaactggaccggcgtgacgcagaacgggggttcctcagcgtgcaagcggggaccgaacaactcgttcttctcccggcttaactggctgtacaagagcggcaacacataccccatgctgaacgtgaccatgcccaactctgacgactttgataagctgtacatctggggagtgcaccacccatcgactgaccgcgaacagaccaacctgtacatccaagcctccggcaaaatcattgtgtccaccaagagatcgcagcagaccatcattccgaacattggcagtcggccatgggtcagaggactgtcgtccagaatcagcatctactggaccattgtgaagcccggcgacatcctcatcataaactcgaacggcaacctgatcgccccacggggatatttcaagatccagaccggaaagagctccgtgatgaagtccgatgccccgattgggacttgcaactccgagtgcatcacccccaacggctccatccccaacgacaaacccttccaaaacgtgaaccgcatcacttacggagcctgcccgcattacatcaagcagaacacgctcaagctcgccactgggatgcgcaacattcctgagcgccaaacccggggaatcttcggagccattgcgggcttcatcgaaaacggctgggaagggatggtcaacggttggtacggttttagacaccaaaattccgaaggaatcggacaggctgccgatctcaaatcaacccaggccgccatcaaccagatcaacgggaagctgaacagagtgatcgaaaagaccaacgaaaagttccatcagattgagaaggagttttccgaagtcgagggacggattcaagacctggagcgctacgtggaggacaccaagattgatctgtggagctacaacgcagagctcctggtcgcgctggaaaatcagcacaccattgacttgaccgactcggagatgaacaagctgtttgaaaagaccaggaagcagctgcgggaaaacgccgaagatatggggaacggatgcttcaaaatctaccataagtgcgacaatagctgcatggaaagcattaggaacgggacttacgaccataacgagtacagagatgaagcagtgaacaacagattccagatcaagtccgtggaactcaagagcggttacaaggactggatcctctggatttcgttcgctatttcgtgcttcctgctgtgcgccatctggatgggattcgtcatctgggcctgccagaagggcaacatccggtgcaacatttgtatttga

SEQ ID NO: 9 - protein, 566 aa, Influenza A virus
  REGION 1..566: note = >ALX30415.1 hemagglutinin [Influenza A virus (A/swine/Italy/240849/2015(H3N2))] EUH3-4
MKTVIALSYVFCLVFGQDFPGKGNNTATLCLGHHAVPNGTLVKTITDDQIEVTNATELVQNFSMGKICKNPHRILDGANCTLVDSLLGDPHCDGFQNEKWDLFIERSRAFSNCYPYDVPEYTSLRSLIASSGTLEFTNENFNWTGVTQNGGSSACKRGPNNSFFSRLNWLYKSGNTYPMLNVTMPNSDDFDKLYIWGVHHPSTDREQTNLYIQASGKIIVSTKRSQQTIIPNIGSRPWVRGLSSRISIYWTIVKPGDILIINSNGNLIAPRGYFKIQTGKSSVMKSDAPIGTCNSECITPNGSIPNDKPFQNVNRITYGACPHYIKQNTLKLATGMRNIPERQTRGIFGAIAGFIENGWEGMVNGWYGFRHQNSEGIGQAADLKSTQAAINQINGKLNRVIEKTNEKFHQIEKEFSEVEGRIQDLERYVEDTKIDLWSYNAELLVALENQHTIDLTDSEMNKLFEKTRKQLRENAEDMGNGCFKIYHKCDNSCMESIRNGTYDHNEYRDEAVNNRFQIKSVELKSGYKDWILWISFAISCFLLCAIWMGFVIWACQKGNIRCNICI

SEQ ID NO: 10 - DNA, 1701 bp, Influenza A virus
  source 1..1701: mol_type = other DNA
atgaaggcaatactagtagttctgctacatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggacatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaatatacatccgatcacaattggaaaatgtccaaaatatgtaaaaagcacaaaattgagactggccacaggattgaggaacgtcccgtctattcaatctagaggcctatttggggccattgccggtttcattgaaggggggtggacagggatggtagatggatggtacggttatcaccatcaaaatgagcaggggtcaggatatgcagccgacctgaagagcacacagcatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctagaatcaacaaggatttaccagattttggcgatctattcaactgtcgccagttcattggtactggtagtctccctgggggcaatcagtttctggatgtgctctaatgggtctctacagtgtagaatatgtatttaa

SEQ ID NO: 11 - DNA, 1701 bp, synthetic construct
  misc_feature 1..1701: note = >EUH1-11 Swine optimized sequence
  source 1..1701: mol_type = other DNA
atgaaggctattctggtggtcctgctccacacttttgccaccgcgaacgcagataccctctgcatcggataccacgccaacaattccaccgatactgtggataccgtgctggaaaagaacgtgaccgtgactcactcagtgaacctcctggaagataagcacaacggaaagctttgcaagcttcggggagtggctccgttgcatctgggaaagtgcaacatcgccggatggatcctgggcaaccccgagtgcgagagcctgtccaccgcctcctcctggtcttacattgtggaaacctcctcgtccgataacggcacatgctatccgggggacttcattgactacgaagaactgagggagcaactcagctccgtgagctcctttgagcgcttcgaaatcttccctaagacctcatcctggcccaaccacgactccaacaagggagtgactgcagcgtgtccgcacgctggagccaagtcattctacaagaacctgatctggctggtcaaaaagggaaactcgtaccccaagctgtctaagtcctacatcaatgacaaggggaaggaagtgctggtgctgtggggaatccaccacccttccacctccgccgaccagcagtcgctgtaccagaacgctgatgcgtacgtgttcgtcggcactagccgctactccaagaagttcaagccagaaattgccatccggcccaaagtccgcgatcaggaggggcggatgaactactactggactctcgtggaacccggggataagatcacgttcgaagctaccggcaacctcgtggtgcctcgctacgccttcgcaatggagaggaatgctggaagcggtatcatcatttcggacactcctgtgcatgactgcaatacgacttgtcagactccgaagggagcgatcaacacttccctcccgttccaaaacatccaccccattacaattggcaaatgtcctaaatacgtgaagtcgaccaagctccggcttgcaactgggctgcgcaatgtgcctagcattcagtcaaggggcctatttggggccatcgccggtttcatcgagggaggatggaccggaatggtggacggttggtatggctaccaccaccaaaacgaacagggctccggttacgccgccgacctgaagtccacccagcacgccattgacgaaatcaccaacaaggtcaactccgtcatcgagaagatgaacacccagttcactgccgtgggaaaggagttcaaccatctggagaagagaattgagaacctcaataagaaagtggacgacggcttcctggacatctggacctacaacgccgagctgctggtcctgctggaaaacgagaggaccctggactaccacgactcgaacgtgaagaacctgtacgagaaggtccgatcacaactgaagaacaacgccaaggaaatcggaaatgggtgcttcgagttctaccacaaatgcgacaatacttgcatggaatccgtgaagaatggcacctacgattaccctaagtattccgaggaagcgaagctgaaccgggaagagattgacggcgtgaagctcgaatcaacccggatctaccagatccttgcgatctactccaccgtggccagctcactcgtgctcgtggtgtccctgggtgccatctccttctggatgtgtagcaacggcagcctccagtgccgcatctgtatctga

SEQ ID NO: 12 - protein, 566 aa, Influenza A virus
  REGION 1..566: note = >AFR76205.1 hemagglutinin [Influenza A virus (A/swine/England/373/2010(H1N1) EUH1-11
MKAILVVLLHTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQHAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 13 - DNA, 1410 bp, Influenza A virus
  source 1..1410: mol_type = other DNA
atgaatccaaatcagaagataataacaattggctctgtttctctcgtcattgcaacattatgcttcttaatgcagatggccatcctaataactactgtaaaattacatttcaaacaatatgagtgcggcttccctgcgaacaaccaagtaataacatgtgagccaacagtaatagaaaggaacacaacagagatagtgtacttaactaacaccaccatagagaaagaaacatgccacaaaacagtggaatacaggaattggtcaaagcctcaatgcaaaataacaggctttgcacctttttccaaggacaattcaattcgactttctgctggtggggacatatgggtgacgagggaaccttacgtgtcatgcgagcctggcaaatgttatcagtttgcactcgggcaagggaccacactagacaataaacattcaaacgatacaatacatgacagaaccccctatcgaactctattgatgaatgaattgggtgtcccatttcatttagggacaagacaagtgtgtattgcatggtccagctcaagttgttatgatgggaaagcatggttgcatgtctgtatcactggacatgataaaaatgcaactgccagtttcatttacgatggtagacttgtagatagcattggttcatggtctaaaaatatacttagaacccaggaatcagaatgcgtttgcatcaatggggtctgtacagtagtaatgactgatggaagtgcttcgggaagagctgatactaaaatactattcattgaagaagggaaaattgttcatattagcccattagcggggagtgcacagcatgtggaggagtgctcctgttatccccgatatcctggcgtaaggtgtatctgcagagacaactggaaaggctctaacagacccgttgtggatataaatatagaagattatagcattgattccagttatgtgtgttcagggcttgttggcgacacacccagaatcaatgacggatccagtagtagctactgccgtgatcctaacaacgaaaaaggaaatcacggagtgaagggctgggcttttgacgatggaaatgatgtgtggatgggaagaacgatcaacgaagattcacgctcaggttatgaaacattcaaagtcattggtggttggtccactcctaattccaaattgcagataaataggcaagtaatagttgatagcaacaataggtcaggttattctggtgttttctccgttgaaggcaaaagctgcatcaatagatgtttctacgtggagttgataagaggaagaaggtcagaagcgcgagtatggtggacctcaaacagtattgttgtattttgtggcacttcaggtacctatggaacaggctcatggcctgatggagcagacatcaacctcatgcctatatga

SEQ ID NO: 14 - DNA, 1410 bp, synthetic construct
  misc_feature 1..1410: note = >EUN2-6 Swine optimized sequence
  source 1..1410: mol_type = other DNA
atgaatccgaaccagaaaattatcaccattgggtccgtcagccttgtgattgccactctgtgctttctgatgcaaatggccatcctcattaccactgtgaagctgcatttcaagcagtacgaatgcggcttccccgcgaacaaccaggtcatcacttgcgagccaaccgtgattgagcggaacactaccgaaattgtatacctcacgaacactaccattgaaaaggagacttgccacaagaccgtggaataccggaactggtccaagcctcagtgcaagattaccgggttcgccccattctccaaggacaatagcatccgcctttcggccggaggagatatttgggtgacgcgcgaaccctacgtcagctgcgaacccgggaagtgttaccagttcgcactgggccagggaaccaccctggataataagcacagcaacgacaccatccacgaccggactccctaccgcaccctcctgatgaatgagctcggcgtgccgttccacctgggtaccagacaggtctgcatcgcctggtcgtcctcctcatgctacgatgggaaggcttggctccacgtgtgcataaccggtcatgacaagaacgccacagcctcattcatctacgacggacggctagtggactccatcggctcctggtccaagaacattctgaggacccaggaatccgaatgcgtctgtatcaacggagtgtgcaccgtggtgatgaccgatggctccgcaagcggaagggccgatactaagatcctgttcatcgaggagggaaagattgtgcacatctctcctctcgcgggatcagcccagcacgtggaagaatgttcctgttaccctcgctacccgggagtgcgctgtatttgccgggacaactggaaaggctccaacaggcctgtggtggacattaatatcgaggactactccatcgatagctcctacgtgtgctccggtctggtcggggacactccaagaatcaacgatggaagctccagcagctactgccgggaccccaataacgaaaaggggaaccacggcgtgaagggttgggcgttcgacgatggaaatgacgtctggatgggacggactatcaacgaggactcacggagcggatacgaaacctttaaggtcatcggcggatggtcgaccccgaacagcaagctgcagatcaaccgccaagtgatcgtggactccaacaaccgctcggggtatagcggggtgttctccgtggagggaaagtcctgcatcaaccggtgcttctacgtcgaactgatcagaggccggagatcagaagcccgcgtgtggtggacctccaactccattgtggtgttttgcggcacttccgggacctatggcactggatcctggcccgatggagcagacatcaacctgatgccaatctga

SEQ ID NO: 15 - protein, 469 aa, Influenza A virus
  REGION 1..469: note = >AKJ82042.1 neuraminidase [Influenza A virus (A/swine/England/61470/2013(H1N2))] EUN2-6
MNPNQKIITIGSVSLVIATLCFLMQMAILITTVKLHFKQYECGFPANNQVITCEPTVIERNTTEIVYLTNTTIEKETCHKTVEYRNWSKPQCKITGFAPFSKDNSIRLSAGGDIWVTREPYVSCEPGKCYQFALGQGTTLDNKHSNDTIHDRTPYRTLLMNELGVPFHLGTRQVCIAWSSSSCYDGKAWLHVCITGHDKNATASFIYDGRLVDSIGSWSKNILRTQESECVCINGVCTVVMTDGSASGRADTKILFIEEGKIVHISPLAGSAQHVEECSCYPRYPGVRCICRDNWKGSNRPVVDINIEDYSIDSSYVCSGLVGDTPRINDGSSSSYCRDPNNEKGNHGVKGWAFDDGNDVWMGRTINEDSRSGYETFKVIGGWSTPNSKLQINRQVIVDSNNRSGYSGVFSVEGKSCINRCFYVELIRGRRSEARVWWTSNSIVVFCGTSGTYGTGSWPDGADINLMPI

SEQ ID NO: 16 - DNA, 1410 bp, Influenza A virus
  misc_feature 1..1410: note = >KU323318.1 Influenza A virus (A/swine/Italy/248147-8/2015(H3N2)) segment 6 neuraminidase (NA) gene, complete cds
  source 1..1410: mol_type = other DNA
atgaatccaaatcaaaagataataacaattggttctgtttctctcactattacaacaatgtgcctcttcttgcagattgccatcctagtaactactataacattgcatttcaagcaatatgaatgcgattcccctgcaaacaaccaagtaataccgtgtgaaccaataataatagaaaaaaacataacaaaaatagtgtatttgaccaataccaccatagagaaagaggtatgcccaaaattaggggaatacaggaattggtcaaaaccacaatgcaagatcacaggatttgcacctttttctaaggacaattcaattcggctctctgcgggtggggccatttgggtcacgagagaaccttatgtgtcatgcgaccctaacaagtgttatcaatttgcattaggacagggaaccacattagataacagacattcaaatgacacaatacatgatagaaccccttttagaaccctgttgatgagtgaattaggtgttccatttcatttgggaaccagacaagtatgcatagcatggtccagttcaagttgtcacgatgggaaagcttggttgcatgtttgtgtcactgggcatgataaaaatgcaactgctagtttcatttatgacggaaagcttgtagacagcatcagttcatggtccaaaaacatactccggactcaggaatcagaatgtgtttgtatcgatggaatctgtacagtggtgatgactgatggaagtgcttcagggaaagctgatactaagatactatttattgaaaaagggaagatcattcatattagtccattgttgggaagtgctcagcatgtagaagaatgttcctgttaccctagataccctgatgtcaggtgtatttgcagggataactggaaaggttcaaataggcccatcgtagacataagaatgaaaaattatagcattggttccagttatatgtgctcaggacttgttggcgacacacccaggaacaatgatgggtctagtaatagcaattgtcggaatcccaataatgaaagaggaaatcatggagtgaaaggttgggcctttgatgatggaaatgacacatggatgggaagaactatcagcaaggactcacgcttaggttacgaaaccttcaaagttgttggtggttggtcccaacccaattccaaatcccagataaatagacaagttattgttgacagcgataatagatcaggttactctggtattttctctgttgaggggaaagattgcattaataggtgtttttatgtggaactaataagaggaaggagacaggaaactagagtgtggtggacttcgaacagtattgttgtgttctgtggcacttctggcacctatgggtcaggctcatggcccgatggagcaaacatcaatttcatgcctgtataa

SEQ ID NO: 17 - DNA, 1410 bp, synthetic construct
  misc_feature 1..1410: note = >EUN2-7 Swine optimized sequence
  source 1..1410: mol_type = other DNA
atgaaccctaatcagaagatcatcacaatcggctccgtgtccctcaccatcaccactatgtgcctcttccttcaaatcgccattctggtcaccaccatcaccctgcatttcaagcagtacgaatgcgactcaccagcgaacaatcaggtcatcccgtgtgaaccgatcattatcgagaagaacatcactaagattgtgtatttgaccaacaccaccattgagaaggaagtctgcccaaagctcggggaataccgcaactggagcaagccccagtgcaagattaccggcttcgccccgttctcaaaagacaactccatccggctgtctgcgggaggagccatctgggtcacgagagaaccgtacgtgtcgtgcgaccccaacaagtgctaccagttcgccctgggacaagggaccaccctggacaaccggcactccaacgacactatacatgataggactcccttccggacccttctcatgagcgaactgggggtccccttccatctcggaaccagacaagtctgcattgcgtggagctcctcctcctgccacgacggaaaagcttggctccacgtctgcgtgacgggccacgacaagaacgccactgcctccttcatctacgatgggaagcttgtggactccatctcgtcatggagcaagaacatcctgcggactcaggagtcagaatgcgtgtgcatcgacggcatttgcaccgtcgtgatgactgacggttcggccagcggaaaggccgacaccaagatcctgttcatcgagaagggaaagatcattcacatctccccacttctgggttccgcgcaacacgtggaagaatgctcctgctacccccgctaccccgatgtccggtgcatctgccgggacaactggaagggttccaaccggccaatcgtggacatccgcatgaagaactattccatcgggtcctcttacatgtgctcgggtctggtcggagatacaccgcgcaacaacgacgggtcctcaaacagcaactgccggaaccctaacaacgagaggggcaaccacggcgtgaaaggatgggctttcgacgacggaaatgacacttggatggggagaaccattagcaaggatagcaggcttggctacgaaaccttcaaggtcgtgggcggttggtcgcagccgaactcgaagtcgcagatcaacagacaggtcatcgtggactcggataacagatcggggtactcagggatcttctccgtggagggaaaggactgcatcaatagatgtttctacgtcgagctgattcgaggccggagacaggaaacccgcgtatggtggacgtccaactcaatcgtcgtgttttgcggtacctccggaacttacggctcgggatcatggcccgatggagccaatattaacttcatgcctgtgtga

SEQ ID NO: 18 - protein, 469 aa, Influenza A virus
  REGION 1..469: note = >ALX30429.1 neuraminidase [Influenza A virus (A/swine/Italy/248147-8/2015(H3N2))] EUN2-7
MNPNQKIITIGSVSLTITTMCLFLQIAILVTTITLHFKQYECDSPANNQVIPCEPIIIEKNITKIVYLTNTTIEKEVCPKLGEYRNWSKPQCKITGFAPFSKDNSIRLSAGGAIWVTREPYVSCDPNKCYQFALGQGTTLDNRHSNDTIHDRTPFRTLLMSELGVPFHLGTRQVCIAWSSSSCHDGKAWLHVCVTGHDKNATASFIYDGKLVDSISSWSKNILRTQESECVCIDGICTVVMTDGSASGKADTKILFIEKGKIIHISPLLGSAQHVEECSCYPRYPDVRCICRDNWKGSNRPIVDIRMKNYSIGSSYMCSGLVGDTPRNNDGSSNSNCRNPNNERGNHGVKGWAFDDGNDTWMGRTISKDSRLGYETFKVVGGWSQPNSKSQINRQVIVDSDNRSGYSGIFSVEGKDCINRCFYVELIRGRRQETRVWWTSNSIVVFCGTSGTYGSGSWPDGANINFMPV

SEQ ID NO: 19 - DNA, 1410 bp, Influenza A virus
  misc_feature 1..1410: note = >KU323247.1 Influenza A virus (A/swine/Italy/179057/2015(H1N1)) segment 6 neuraminidase (NA) gene, complete cds
  source 1..1410: mol_type = other DNA
atgaatccaaaccaaaagataataaccattggttcggtctgtatgacaattggaatggctaacttaatattacaaattggaaacataatctcaatatggattagccactcaattcaacttgggaatcaaagtcagattgaaacatgcaatcaaagcgtcattacttatgaaaacaacacttgggtaaatcagacatatgttaacatcagcaacaccaactttgctgctggacagtcagtggtttccgcgaaattagcgggcaattcctccctctgccctgttagtggatgggctatatacagtaaagacaacagtgtaagaatcggttccaagggggatgtgtttgtcataagggaaccattcatatcatgctcccccttagaatgcagaaccttcttcttgactcaaggggccttgctaaatgacaaacattccaatggaaccattaaagataggagcccatatcgaaccctgatgagctgtcctattggtgaagttccctctccatacaactcaagatttgagtcggtcgcttggtcagcaagtgcttgtcacgatggcatcaattggctaacaatcggaatttctggcccagacagtggggcagtggctgtattaaagtacaatggcataataacagacactatcaagagttggaaaaacaatatattgagaacacaagagtctgaatgtgcatgtgtaaatggttcttgctttaccataatgaccgatggaccaagtgatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaaggattttcattcaaatatggcaatggtgtttggatagggagaactaaaagcattagttcaagaaaaggttttgagatgatttgggatccaaatggatggactgggacagacaaaaacttctcaataaagcaagatatcataggaataaatgagtggtcaggatacagcgggagttttgttcagcatccagaactaacagggctgaattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaagtaa

SEQ ID NO: 20 - DNA, 1410 bp, synthetic construct
  misc_feature 1..1410: note = >EUN1-4 Swine optimized sequence
  source 1..1410: mol_type = other DNA
atgaaccctaaccagaagattattaccatcggatccgtgtgcatgaccatcggaatggccaacctgatcctgcaaatcggaaacatcatctccatctggattagccatagcattcagcttggaaaccagagccagatcgagacttgtaaccagtccgtgatcacctacgagaacaacacctgggtcaaccagacttacgtcaatatcagcaacaccaacttcgctgcggggcagtccgtggtgtccgcaaagctggccggcaacagctccctgtgccccgtgtccggatgggcgatctactccaaggataacagcgtgcgcatcgggtccaaaggggacgtgttcgtcatccgcgaaccattcatttcttgctcccccttggaatgtcggaccttcttcctgacccaaggggcgttgctcaacgacaagcacagcaacggaaccatcaaagatcggtcgccctaccgcactctgatgtcgtgccctatcggcgaagtgccatccccctacaactcacgcttcgagtccgtggcctggtccgcttccgcctgccacgatggaatcaactggctcacaatcggcatctccggcccggactcgggagccgtggccgtgctgaagtacaatggtattattactgacactatcaagtcgtggaagaacaatattctccggactcaagaatctgaatgcgcctgcgtgaacggttcctgcttcactatcatgaccgacggcccttccgatggacaggcctcatacaagatcttccggatcgagaagggaaagatcgtgaagtccgtcgagatgaacgcaccgaactaccattatgaggaatgctcgtgctacccggactcctcggaaattacttgcgtgtgccgcgacaattggcacgggtccaacaggccctgggtgtccttcaaccaaaacctggagtaccagatcggttacatctgctccgggatttttggagacaaccctagacctaacgacaagaccggctcatgcggacctgtgtcctccaacggagccaacggcgtgaagggattctcgttcaaatatgggaacggcgtctggataggtcggaccaagtccatctcgtcacggaagggctttgaaatgatttgggacccgaacggttggaccggaaccgacaagaacttcagcatcaagcaggacattatcggcattaacgagtggagcggatactcgggcagcttcgtccagcacccggaacttacgggcctcaattgtattaggccctgtttttgggtcgagctgattagagggcgccccaaggaaaacaccatctggaccagcggctccagcatctcattctgcggagtgaactccgacaccgtgggctggtcgtggcccgacggtgccgagctgccgttcaccatcgataaatga

SEQ ID NO: 21 - protein, 469 aa, Influenza A virus
  REGION 1..469: note = >ALX30323.1 neuraminidase [Influenza A virus (A/swine/Italy/179057/2015(H1N1))] EUN1-4
MNPNQKIITIGSVCMTIGMANLILQIGNIISIWISHSIQLGNQSQIETCNQSVITYENNTWVNQTYVNISNTNFAAGQSVVSAKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWKNNILRTQESECACVNGSCFTIMTDGPSDGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRKGFEMIWDPNGWTGTDKNFSIKQDIIGINEWSGYSGSFVQHPELTGLNCIRPCFWVELIRGRPKENTIWTSGSSISFCGVNSDTVGWSWPDGAELPFTIDK

SEQ ID NO: 22 - DNA, 1410 bp, Influenza A virus
  misc_feature 1..1410: note = >KR700532.1 Influenza A virus (A/swine/Italy/28762-3/2013(H1N1)) segment 6 neuraminidase (NA) gene, complete cds
  source 1..1410: mol_type = other DNA
atgaacccaaatcagaagataataatcattagttcaatctgtatgacaaatggaattgctagcttgatattacaaattgggaacataatatcaatatggattagccattcaattcaaattgagaacccaaaccagaccgaaccatgcaatcaaagcgttattatttacgaaaacaacacatgggtaaatcaaacgtatgttaacatcagcaacaataattttgttgttgaacagacagtggtttcaatgaaattagcgggcagttcttctctctgccctgttagtggatgggctatatacagtaaagataacagtgtaagaatcggttccaaaggggatgtgtttgtcataagagagccattcatctcatgctcccatttggaatgtagaaccttcttcttaactcaaggggccctactgaatgataaacattctaatggaaccgttaaagacagaagcccctatcgaaccctgatgagctgtcctattggtgaagtcccctctccatacaactcaaaatttgagtcagttgcttggtcagcaagtgcttgccatgatggcaccagttggttgacaattgggatttctggtccagacaatggagcagtggctgtgttgaaatacaatgacataataacagacactatcaagagttggaaaaacaacatattgagaacacaagaatctgaatgtgcatgtttgaatggttcttgctttactgtaatgaccgatggaccaagtaatgggcaggcctcatacaagatcttcaaaatagaaaaggggaaagtagtcaaatcagtcgagttgaatgctcctaattatcactatgaggaatgttcctgttatcctgattctggtgaaatcatatgtgtatgcagggacaattggcatggctcgaatcgaccatgggtgtctttcaatcagaatctggagtatcagataggatacatatgcagtggggttctcggagacaatccgcgccctaatgatagaacaggcagttgtggtccagtatcatctcatggagcaaatggggtaaaagggttttcgtttaaatacggcaatggaatttggatagggagaactaaaagcactattacaaggagtggttttgagatgatttgggacccaaacggatggactggaacagacaataatttctcagtgaagcaagatatcgtaggaataactaactggtcaggatacagcgggagttttgtccaacatccagaattaaccggattggattgtattagaccttgcttctgggttgaactaatcagagggagacccaaagagaacacaatctggactagcggaagcagcatatccttttgtggtgtaaatagtgacactgtgggttggtcttggccagacggtgctgagttgccatttaccattgacaagtaa

SEQ ID NO: 23 - DNA, 1410 bp, synthetic construct
  misc_feature 1..1410: note = >EUN1-2 Swine optimized sequence
  source 1..1410: mol_type = other DNA
atgaatccgaatcagaagatcattatcatctcctccatttgtatgactaacggaatcgcatctctgatcttgcaaattgggaacatcattagcatctggatctcccactcgatccaaatcgagaacccgaaccagaccgagccctgcaaccagagcgtgatcatatacgaaaacaatacctgggtgaaccagacctatgtcaatatctcgaacaacaacttcgtggtggagcagactgtcgtgtccatgaagctcgccgggtccagctcgttgtgccctgtctccggctgggctatctacagcaaagacaacagcgtcagaatcggcagcaagggagatgtgttcgtgatccgcgagcctttcatctcctgctcacacctggaatgccggacctttttcctgacacaaggcgccctgctcaatgacaagcactcgaacgggaccgtgaaggacagaagcccctacagaaccctgatgtcctgtccgattggagaagtgccctccccgtataactcaaagttcgaatccgtggcttggtcagcatccgcgtgccatgacgggaccagctggctgactatcggaatctccggtcctgacaacggcgcggtcgccgtgctcaagtacaacgacattatcactgacaccatcaagtcctggaaaaacaacatcctccggacccaagagtccgagtgcgcctgcctgaacggttcgtgtttcaccgtgatgaccgatggaccaagcaacggacaagcctcgtacaagattttcaagatcgaaaagggaaaagtggtcaagtccgtggagctgaacgcccctaactaccactacgaagagtgttcatgctaccctgactccggcgaaattatctgcgtgtgtcgggataactggcacggatccaaccgcccctgggtcagcttcaaccagaacctggaatatcagatcggctacatttgctccggcgtgctgggggacaacccgaggccgaatgaccgcactggttcctgcggacccgtgtcatcccacggggcgaacggagtgaagggtttctctttcaaatacggaaacggcatctggattggcagaaccaagagcactatcacccggagcggatttgagatgatctgggaccccaacggatggaccggtaccgataacaacttttcggtcaagcaggatattgtgggcatcactaactggtcgggatactccggctcgttcgtgcagcatcccgagctgactggcctggactgcattcgcccgtgcttctgggtggaacttatcaggggtcggcctaaggagaacactatctggacctccggatcgtcgatttccttctgcggcgtgaactcagataccgtgggatggtcctggccggacggggccgagcttccattcacgattgataagtga

SEQ ID NO: 24 - protein, 469 aa, Influenza A virus
  REGION 1..469: note = >AKJ81669.1 neuraminidase [Influenza A virus (A/swine/Italy/28762-3/2013(H1N1))] EUN1-2
MNPNQKIIIISSICMTNGIASLILQIGNIISIWISHSIQIENPNQTEPCNQSVIIYENNTWVNQTYVNISNNNFVVEQTVVSMKLAGSSSLCPVSGWAIYSKDNSVRIGSKGDVFVIREPFISCSHLECRTFFLTQGALLNDKHSNGTVKDRSPYRTLMSCPIGEVPSPYNSKFESVAWSASACHDGTSWLTIGISGPDNGAVAVLKYNDIITDTIKSWKNNILRTQESECACLNGSCFTVMTDGPSNGQASYKIFKIEKGKVVKSVELNAPNYHYEECSCYPDSGEIICVCRDNWHGSNRPWVSFNQNLEYQIGYICSGVLGDNPRPNDRTGSCGPVSSHGANGVKGFSFKYGNGIWIGRTKSTITRSGFEMIWDPNGWTGTDNNFSVKQDIVGITNWSGYSGSFVQHPELTGLDCIRPCFWVELIRGRPKENTIWTSGSSISFCGVNSDTVGWSWPDGAELPFTIDK

SEQ ID NO: 25 - DNA, 13 bp, synthetic construct
  misc_feature 1..13: note = 5` flanking sequence of open reading frame
  source 1..13: mol_type = other DNA
ggcgcgccgcacc

(19) United States
(12) Patent Application Publication
(10) Pub. No.: US 2026/0217771 A1
(43) Pub. Date: Jul. 30, 2026

(54) SWINE INFLUENZA A VIRUS VACCINE COMPRISING A NUCLEIC ACID CONSTRUCT HAVING A SPECIFIC ORDER OF GENES

(71) Applicant: Intervet Inc., Rahway, NJ (US)

(72) Inventors: Mark A. Mogler, Ames, IA (US); Basav Hangalapura Nagaraj, Boxmeer (NL)

(73) Assignee: Intervet Inc., Rahway, NJ (US)

(21) Appl. No.: 19/644,538

(22) Filed: Apr. 10, 2026

Related U.S. Application Data
(63) Continuation of application No. 18/010,418, filed on Jun. 18, 2021, now Pat. No. 12,630,592.

(30) Foreign Application Priority Data
Jun. 19, 2020 (EP) 20181094.2

(51) Int. Cl. C07K 14/11; A61K 39/00; A61P 31/16; C12N 15/86

(52) U.S. Cl. CPC C07K 14/11; A61P 31/16; C12N 15/86; A61K 2039/552; A61K 2039/55566; A61K 2039/70

(57) ABSTRACT

The present invention relates to a nucleic acid construct that encodes, in this order, a first Swine influenza A (IAV-S) hemagglutinin (HA) antigen of the Scot/94 lineage and a second Swine influenza A (IAV-S) hemagglutinin (HA) antigen of the Eurasian avian-like (EA) lineage, and a nucleic acid construct that encodes, in this order, a first IAV-S HA antigen of the Gent/84 lineage and a second IAV-S HA antigen of pandemic09 (pdm09) lineage. In other embodiments, the present invention relates to RNA replicon particles comprising one or both nucleic acid constructs, an immunogenic composition, such as a vaccine, which may be used against influenza A virus infections, and comprising the replicon particles. Further provided are methods of making the vaccine and use of the vaccine.

### CROSS-REFERENCE TO RELATED APPLICATIONS

This application is a continuation of U.S. patent application Ser. No. 18/010,418 filed Dec. 14, 2022; which is a National Stage application of International Patent Application No. PCT/EP2021/066546, filed Jun. 18, 2021, which claims priority to European Patent Application No. EP 20181094.2, filed Jun. 19, 2020.

### Reference to Sequence Listing Submitted Electronically

The instant application contains an electronic sequence listing which has been submitted in XML format via the Patent Center, the entire content of which is hereby incorporated by reference in its entirety. The Sequence Listing XML file submitted via the Patent Center is entitled 24970-US-CNT.xml and was created on Mar. 31, 2026 and is 60,399 bytes in size.

The present invention relates to a nucleic acid construct that encodes, in this order, a first Swine influenza A (IAV-S) hemagglutinin (HA) antigen of the Scot/94 lineage and a second Swine influenza A (IAV-S) hemagglutinin (HA) antigen of the Eurasian avian-like (EA) lineage, and a nucleic acid construct that encodes, in this order, a first IAV-S HA antigen of the Gent/84 lineage and a second IAV-S HA antigen of pandemic09 (pdm09) lineage. In other embodiments, the present invention relates to RNA replicon particles comprising one or both nucleic acid constructs, an immunogenic composition, such as a vaccine, which may be used against influenza A virus infection, and comprising the replicon particles. Further provided are methods of making the vaccine and use of the vaccine.

### BACKGROUND

Influenza A viruses (IAV) create a significant burden on human and animal health, worldwide. IAV is categorized into different subtypes based on its viral surface glycoproteins, hemagglutinin (HA) and neuraminidase (NA). IAV infects poultry, pigs, horses, cats, dogs, marine mammals (e.g., whales), bats and humans. Wild waterfowl and shorebirds (ducks, geese, swans and gulls) are the natural reservoirs and they can be infected with 16 different HA and 9 different NA subtypes [Webster et al., Microbiol Rev 56:152-179 (1992)].

Influenza A virus in swine (IAV-S) is a serious respiratory pathogen of domestic pigs that has proven to be economically costly, particularly to the livestock industry, worldwide [Holtkamp et al., The American Association of Swine Veterinarians Annual Meeting (2007)]. It is characterized by a sudden onset of respiratory illness, and is usually accompanied by anorexia, lethargy, and fever. In addition to the clinical complications associated with IAV-S in production animals, there have been published reports implicating swine in the transfer of influenza viruses to humans [Myers K P, Olsen C W, Gray G C. Clin Infect Dis 2007; 44 (8): 1084-8, Krueger and Gray, Curr Top Microbiol Immunol 370:201-225 (2013)], which represents a significant public health threat providing an even greater incentive to control IAV in swine herds.

In response to this problem, many swine farmers now vaccinate their pigs against IAV-S employing commercially available vaccines. However, controlling IAV-S with the conventional vaccines is difficult because many diverse IAV-S strains co-circulate in the field and continue to evolve [Gao et al., J Gen Virol 98 (8): 2001-2010 (2017)]. The diversity and mutability of IAV-S are caused by the virus's genetic structure. Like other influenza A viruses, IAV-S has genes encoded on eight segments of RNA and a genome replication machinery that introduces frequent mutations. These genetic characteristics enable IAV-S to make rapid adaptions, including escape from existing neutralizing antibodies induced by exposure to previous strains. Consequently, inactivated virus IAV-S vaccines that are commercially available in the USA market have proven inadequate despite comprising up to five different IAV-S strains due to newly emerging strains that arise as a consequence of the continuous antigenic drift and or shift.

Classification of influenza A viruses starts with subtyping of HA and NA, the two major glycoproteins on the virus surface. HA protein mediates attachment and fusion of the virus to host cells. Neuraminidase is an enzyme that functions in the final stage of the influenza virus replication cycle by cleaving newly formed viral particles from the host cell, thereby enabling the new progeny virus to spread and infect other cells. Recent research has shown that NA immunity only can play a supplemental and/or complementary role to the more critical HA immunity [Nayak et al., J Virol 84 (5): 2408-2420 (2010); Pavlova et al., Vaccine 27 (5): 773-785 (2009); Sylte et al., Vaccine 25 (19): 3763-72 (2007)]. Indeed, it appears that in the absence of a hemagglutinin antigen, a neuraminidase influenza A virus vaccine is not potent enough to either protect against influenza A infection or protect against an influenza A virus induced disease.

Whereas human influenza A usually has 1 or 2 dominant strains circulating globally during a given influenza season, many more strains of IAV-S co-circulate simultaneously, with these strains differ between geographic regions. Similarly, IAV-S strains are also antigenically variable, but mainly contain an H1 or H3 subtype of HA, and a N1 or N2 subtype of NA. Within each HA and NA subtype of IAV-S there is further phylogenetic diversity.

In the US swine population there are four predominant phylogenetic clusters of H1 (gamma, delta1, delta2, pandemic), two predominant clusters of H3 (cluster IV and human-like), two predominant clusters of N1 (classic, pandemic), and two predominant clusters of N2 (N2-1998 and N2-2002). [See, Anderson et al., Influenza and other Respiratory Viruses 7 (Suppl. 4); 42-51 (2013); and Anderson et al., mSphere 1 (6) e00275-16:1-14 (2016)].

In Europe there are three major lineages of H1 (Eurasian-avian like H1, Scotland/410440/1994-like H1 and pandemic 2009 like H1), one major lineage of H3 (Gent/1/1984-like H3), two major lineages of N1 (Eurasian Avian-like N1, Pandemic 2009 like N1), two major lineages of N2 (Gent/1/1984-like N2, Scotland/410440/1994-like N2) and two minor lineages of N2 (Italy/4675/2003 like N2, Human seasonal like N2) [Watson et al., J. Virol., 89:9920-9931 (2015); doi: 10.1128/JVI.00840-15].

Vaccination against IAV-S represents the best option for decreasing clinical complications in swine as well as decreasing opportunities for additional reassortment and zoonotic spread from swine to humans. Until recently, the only vaccines available for widespread use are inactivated vaccines prepared from influenza viruses grown in embryonated eggs, but their supply is limited, in large part by a paucity of specific pathogen-free eggs, and the need for new approaches to influenza vaccines is well recognized.

With conventional inactivated virus IAV-S vaccines the choice of viral strains is based on HA antigen properties. IAV-S vaccines that induce HA inhibiting (HI) antibody titers protect pigs against experimental infection with an antigenically similar strain [Kyriakis et al., Vet Microbiol 144 (1-2): 67-74 (2010)]. However, relatively rapid genetic drift of the HA genes allows new strains to emerge that are not functionally inhibited by the vaccine-induced HA antibodies.

As a consequence, commercially available vaccines often do not protect against new and emerging virus subtypes/clusters, and offer only limited protection against heterosubtypic challenge, since the antigens do not match all contemporary strains circulating in the field [Lee et al., Can J Vet Res 71 (3): 207-12 (2007); Vincent et al., Vaccine 28 (15): 2782-2787 (2010)]. Thus, such vaccines must be periodically updated to match currently circulating strains.

Therefore, there is a need in the art to develop novel IAV-S vaccines that are safe, effective, and can be rapidly altered to antigenically match an emerging strain.

Because most viruses, such as influenza viruses, are relatively simple structures, the use of a single antigen from their antigen profile can sometimes suffice to generate a protective immune response. Such a subunit vaccine can be manufactured by extraction from the virus or its culture, or by the recombinant expression of the specific antigen. Alternatively, the viral antigen can be delivered to a target animal and expressed inside it, by a live recombinant carrier micro-organism that acts as a vector. Vectors can be live attenuated or non-live. A number of vector-based strategies have been employed through the years for vaccines in an effort to protect against certain pathogens.

A variation on the use of viral vector vaccines is the use of vaccines based on replicon particles [RP; see Lundstrom, 2014, Vaccines, vol. 6, p. 2392-2415]. These are virus-like particles but comprise a defective viral genome and typically, a heterologous gene. These replicon particles typically comprise RNA packaged in particles (i.e., they are encapsidated) such that they are able to enter a target animal host cell and perform one round of viral genome amplification without the ability to form new particles. The replicon particle does not propagate from the infected cell, as it lacks the necessary structural protein-coding sequence(s). As such, they are more similar to wild-type virus (e.g. in terms of tropism) than other replicon vaccines such as naked RNA vaccines, or vaccines comprising RNA launched from a DNA plasmid.

The genome of the RP's typically expresses a heterologous gene encoding an immunoprotective antigen. Most widely used and most extensively studied are alphavirus RNA replicon particles [Vander Veen et al., 2012, Anim. Health. Res. Rev., vol. 13, p. 1-9; and: Kamrud et al., 2010, J. Gen. Virol., vol. 91, p. 1723-1727], which are therefore preferred for practical reasons, and which have been developed from viral genomes by replacing the structural protein genes with heterologous genes. The resulting RNAs, called replicons, are capable of directing their own replication and express high levels of the heterologous gene when they are introduced into the cytoplasm of host cells. Since these replicons lack the alphavirus structural protein genes, they are incapable of forming virions and spreading to adjacent cells. However, replicons can be efficiently packaged into virus replicon particles (RPs) by introducing them into cells where the structural proteins are provided in trans [Pushko et al., 1997, Virology, vol. 239, p. 389-401].

Also, alphavirus RP's are believed to be somewhat stronger immunopotentiators than other RP's known in the art and based on other viruses such as the bunyavirus. Several Alphavirus species have been used to develop RP vaccines, e.g.: Venezuelan equine encephalitis virus (VEEV) [Pushko et al., 1997, Virology, vol. 239, p. 389-401], Sindbis virus [Bredenbeek et al., 1993, J. of Virol., vol. 67, p. 6439-6446], and Semliki Forest virus [Liljestrom & Garoff, 1991, Biotechnology (NY), vol. 9, p. 1356-1361].

RP vaccines can elicit mucosal and systemic immune responses following immunization of a target animal [Davis et al., 2002, IUBMB Life, vol. 53, p. 209-211]. VEE based RP vaccines are also the basis of several USDA-licensed vaccines, which include: Porcine Epidemic Diarrhea Vaccine, RNA (Product Code 19U5.P1), Swine Influenza Vaccine, RNA (Product Code 19A5.D0), Avian Influenza Vaccine, RNA (Product Code 1905.D0), and Prescription Product, RNA Particle (Product Code 9PP0.00).

Since the RP vector system can be easily manipulated at a molecular level, vaccines can be produced quickly in order to respond to emerging virus subtypes.

Thus, there is an ongoing demand for novel vaccines, which provide for a broad protection against circulating IAV-S strains, in particular for providing broad protection against most or all of the four main IAV-S strains circulating in Europe of EurAsianAvian H1N1, Gent84 H3N2, Scot/94 H1N2 and pandemic2009 H1N1, and which can be rapidly adapted to respond to emerging virus subtypes and antigenic drift.

However, the RP vector system, such as the alphavirus replicon platform, does not allow for the insertion of any desired number of antigens, such as the insertion of all NA and HA genes of the four main circulating IAV-S strains into the replicon vector, in order to achieve broadest protection. Alphavirus vector platforms are typically a three-component system composed of an RNA containing the nonstructural genes with their associated packaging signal and the structural proteins removed and replaced with heterologous gene sequences. Two helper RNAs contains the virus structural proteins without the packaging signal. These three-component, replicon, based systems are limited in the amount of RNA they can package by the volume of the virus capsid [Nanda K. et al., Vol. 390 (2), 2009, 368-373]. This intrinsic limitation of the RP vector system makes it difficult to meet the ongoing demand of providing a vaccine having broad protection against most or all circulating IAV-S strains.

### SUMMARY OF THE INVENTION

In a first aspect of the present invention, it has surprisingly been found that in case of inserting more than one swine influenza A virus hemagglutinin (IAV-S HA) antigen, the position of the genes encoding the HA antigens within the viral genome of an RNA replicon particle greatly impacts the level of induced immunity.

Accordingly, the present invention provides nucleic acid constructs that encode a combination of two IAV-S HA antigens from different lineages in a specific order. These nucleic acid constructs can be used in RNA replicon particles. These RNA replicon particles of the present invention may be used in immunogenic compositions for providing vaccines for use in the prevention of a disease caused by a Swine influenza A virus (IAV-S) in a vaccinated subject (e.g. a human, companion animal or livestock, particularly swine).

In a first embodiment of the present invention, the nucleic acid construct comprises a combination of the IAV-S HA antigen of the Scot/94 lineage and the Eurasian avian-like (EA) lineage, with the IAV-S HA of the Scot/94 lineage placed first (in the order from 5′ to 3′ of the nucleic acid sequence) and the IAV-S HA of the EA lineage placed second. The term “in 5′ to 3′ direction”, also known as: ‘in downstream direction’, is well-known in the field. Together with the terms “in this order” it serves to indicate the relative orientation which the elements that are summed up thereafter need to have in respect of each other, in order to be functional with the gene-expression machinery of a host cell, i.e. in which a RP according to the invention comprising the nucleic acid construct can be replicated and expressed. As the skilled person will realise, in the present case, this direction relates to the nucleic acid strand of a genome that is the ‘coding strand’. The genes may be present in a consecutive order in the 5′ to 3′ direction, i.e. there are no intermediate genes for expression into proteins present in the construct. In that case the nucleic acid construct typically comprises, in the order from 5′ to 3′, the backbone virus nonstructural protein open reading frame, a subgenomic promoter followed by the first HA antigen gene sequence, interstitial sequence, a second subgenomic promoter sequence followed by a second HA antigen gene, and finally the backbone virus 3′ untranslated region.

Thus, the present invention provides a nucleic acid construct comprising, in the order from 5′ to 3′ of the nucleic acid sequence:
a first nucleic acid sequence encoding a first hemagglutinin (HA) antigen of IAV-S of the A/swine/Scotland/410440/1994-like H1ₕᵤN2 (Scot/94) lineage, and
a second nucleic acid sequence encoding a second HA antigen of IAV-S of the Eurasian avian-like H1ₐᵥN1 (EA) lineage

In a second embodiment of the present invention, the nucleic acid construct comprises a combination of the IAV-S HA antigen of the Gent/84 lineage and the pdm09 lineage, with the IAV-S HA of the Gent/84 lineage placed first (in the order from 5′ to 3′ of the nucleic acid sequence) and the IAV-S HA of the pdm09 lineage placed second. Thus, the present invention provides a nucleic acid construct comprising, in the order from 5′ to 3′ of the nucleic acid sequence:

a first nucleic acid sequence encoding a first HA antigen of IAV-S of the A/swine/Gent/1/1984-like H3N2 (Gent/84) lineage, and
a second nucleic acid sequence encoding a second HA antigen of IAV-S of the A (H1N1) pdm09 (pdm09) lineage.

In a second aspect of the present invention, it has surprisingly been found that the swine influenza A virus hemagglutinin (IAV-S HA) of certain strains of the four major circulating IAV-S lineages can provide for improved immunity against IAV-S compared to other strains. In particular, it has been found that specific combinations of IAV-S HA can provide for improved immunity. Thus, such combinations of IAV-S HA can beneficially be used in nucleic acid constructs, which can be included in RNA replicon particles. These RNA replicon particles can be used as immunogenic compositions for providing vaccines that aid in the protection of the vaccinated subject (e.g. a human, companion animal or livestock, particularly swine) against IAV-S, e.g. aid in the prevention of IAV-S virus infection.

Thus, the present invention further provides nucleic acid constructs that encode a combination of two IAV-S HA antigens of specific strains as defined herein.

In a first embodiment, the present invention provides a nucleic acid construct comprising first and second nucleic acid sequences:
a first nucleic acid sequence encoding a first HA antigen of IAV-S of the A/swine/Scotland/410440/1994-like H1ₕᵤN2 (Scot/94) lineage from strain A/swine/Italy/3033-1/2015 (H1N2), and
a second nucleic acid sequence encoding a second HA antigen of IAV-S of the Eurasian avian-like H1ₐᵥN1 (EA) lineage from strain A/swine/Italy/28762-3/2013 (H1N1).

In a second embodiment, the present invention provides a nucleic acid construct for use in the prevention or treatment of a disease caused by a Swine influenza A virus in a subject, the nucleic acid construct comprising first and second nucleic acid sequences:
the first nucleic acid sequence encoding a first hemagglutinin (HA) antigen of a Swine influenza A virus (IAV-S) of the A/swine/Gent/1/1984-like H3N2 (Gent/84) lineage from strain A/swine/Italy/240849/2015 (H3N2), and
a second nucleic acid sequence encoding a second HA antigen of IAV-S of the A (H1N1) pdm09 (pdm09) lineage from strain A/swine/England/373/2010 (H1N1).

In another important embodiment, there is provided an RNA replicon particle comprising the nucleic acid construct of the present invention. Thus, the RNA replicon particle may comprise the nucleic acid construct according to the first or according to the second embodiment.

Encompassed by the present invention are any combinations of the embodiments of the first and second aspects as described herein. Thus, the present invention further provides nucleic acid constructs in which the IAV-S HA antigens are arranged in the specific order as defined in the first aspect and in which the IAV-S antigens are from the specific strains as defined in the second aspect.

In another important aspect, the present invention provides an RNA replicon particle comprising the nucleic acid constructs as described herein.

In another important aspect, the present invention provides an immunogenic composition, comprising the RNA replicon particle as described herein.

In another important aspect, the present invention provides an immunogenic composition comprising a combination of RNA replicon particles, the combination comprising a first RNA replicon particle comprising the nucleic acid construct according to the first embodiment and a second RNA replicon particle comprising the nucleic acid construct according to the second embodiment.

A further embodiment of the present invention relates to a vaccine comprising the immunogenic composition as described herein.

In another important embodiment, the vaccine of the present invention may be used in the prevention or treatment of a disease caused by a Swine influenza A virus in a subject.

In another important embodiment, the present invention provides a method of immunizing a porcine against a swine influenza A virus, the method comprising administering to the porcine an immunologically effective amount of the vaccine of the present invention.

In a third aspect, it has surprisingly been found that a combination of two RNA replicon particles each comprising a nucleic acid construct encoding first and second HA antigens of IAV-S of different lineages can provide for improved immunity against IAV-S.

Thus, the present invention further provides an immunogenic composition comprising first and second RNA replicon particles, the first RNA replicon particle comprising a nucleic acid construct, comprising first and second nucleic acid sequences encoding first and second HA antigens of IAV-S, wherein
the first HA antigen is a of the A/swine/Gent/1/1984-like H3N2 (Gent/84) lineage, and
the second HA antigen is of the A (H1N1) pdm09 (pdm09) lineage, the second RNA replicon particle comprising a nucleic acid construct comprising third and fourth nucleic acid sequences encoding third and fourth HA antigens of IAV-S, wherein
the third HA antigen is of the A/swine/Scotland/410440/1994-like H1ₕᵤN2 (Scot/94) lineage, and
the fourth HA antigen is of the Eurasian avian-like H1ₐᵥN1 (EA) lineage.

Encompassed by the present invention are any combinations of the embodiments of the third aspect with the embodiments of the first and second aspects as described herein. Thus, the present invention further provides replicon particles as described in the third aspect, wherein the nucleic acid constructs encode IAV-S HA antigens, which are arranged in the specific order as defined in the first aspect and/or in which the IAV-S antigens are from the specific strains as defined in the second aspect.

In a fourth aspect, it has surprisingly been found that a nucleic acid construct comprising a specific combination of IAV-S neuraminidase (NA) antigens of three different lineages as described herein can be used for providing immunity against all four major circulating IAV-S lineages.

Thus, the present invention further provides a nucleic acid construct comprising first, second and third nucleic acid sequences encoding first, second and third NA antigens of IAV-S, wherein
the first NA antigen is of the A/swine/Scotland/410440/1994-like H1ₕᵤN2 (Scot/94) lineage,
the second NA antigen is of the A/swine/Gent/1/1984-like H3N2 (Gent/84) lineage, and
the third NA antigen is selected from the A (H1N1) pdm09 (pdm09) lineage or the Eurasian avian-like H1ₐᵥN1 (EA) lineage.

In another important embodiment, the present invention provides an RNA replicon particle comprising the nucleic acid construct as described in the fourth aspect.

In another important embodiment, the present invention provides an immunogenic composition, comprising the RNA replicon particle as described in the fourth aspect.

A further embodiment of the present invention relates to a vaccine comprising the immunogenic composition as described in the fourth aspect.

In another important embodiment, the vaccine as described in the fourth aspect may be used in the prevention or treatment of a disease caused by a Swine influenza A virus in a subject.

In another important embodiment, the present invention provides a method of immunizing a porcine against a swine influenza A virus, the method comprising administering to the porcine an immunologically effective amount of the vaccine as described in the fourth aspect.

In another important aspect, the present invention provides an immunogenic composition comprising a combination of RNA replicon particles, the combination comprising first and second RNA replicon particles according to the third aspect, and a third RNA replicon particle comprising the nucleic acid construct according to the fourth aspect.

Encompassed by the present invention are any combinations of the embodiments of the fourth aspect with the embodiments of the first, second and/or third aspects as described herein. Thus, the present invention further provides replicon particles as described in the third aspect, wherein the nucleic acid constructs encode IAV-S HA antigens, which are arranged in the specific order as defined in the first aspect and/or in which the IAV-S antigens are from the specific strains as defined in the second aspect in combination with replicon particles as described in the fourth aspect.

### BRIEF DESCRIPTION OF THE FIGURES

FIG. 1: Hemagglutination inhibition (HI) antibody titers induced by single-gene RNA particle encoding one HA antigen of EurAsianAvian lineage IAV-S.

FIG. 2: HI antibody titers induced by single-gene RNA particle encoding one HA antigen of Scot 1994 lineage IAV-S.

FIG. 3: HI antibody titers induced by single-gene RNA particle encoding one HA antigen of Pdm 2009 lineage IAV-S.

FIG. 4: HI antibody titers induced by single-gene RNA particle encoding one HA antigen of Gent 1984 lineage IAV-S.

FIG. 5: HI antibody titers induced by dual-gene RNA particle encoding one HA antigen of EurAsianAvian (EUHA1-2, EUHA1-3 & EUHA1-5) and another of Scot1994 (EUHA1-15 or EUHA1-17) lineage IAV-S strains in different combinations.

FIG. 6: HI antibody titers induced by dual-gene RNA particle encoding one HA antigen of pandemic (EUHA1-11) and another of Gent1984 (EUHA3-4) or one HA antigen of Scot1994 (EUH1-15, EUHA1-17) and another of EurAsianAvian (EUHA1-3 & EUHA1-5) lineage IAS strains in two different positions.

FIG. 7: Neuraminidase inhibition (NI) antibody titers induced by single-gene RNA particle encoding one NA antigen of EurAsianAvian (EA) lineage IAV-S.

FIG. 8: (NI) antibody titers induced by single-gene RNA particle encoding one NA antigen of Pdm09 lineage IAV-S.

FIG. 9: (NI) antibody titers induced by single-gene RNA particle encoding one NA antigen of Scot/94 lineage IAV-S.

FIG. 10: (NI) antibody titers induced by single-gene RNA particle encoding one NA antigen of Gent/84 lineage IAV-S.

FIG. 11: NI antibody titers induced by dual-gene RNA particles encoding one NA antigen of EurAsianAvian (EUNA1-2) and another NA of Gent1984 (EUNA2-7) or triple-gene RNA particles encoding one NA antigen each of EurAsianAvian (EUNA1-2), Gent1984 (EUNA2-7) and Scot1994 (EUHNA2-6) lineage IAS strains in different positions.

FIG. 12: NI antibody titers induced by dual-gene RNA particles encoding one NA antigen of EurAsianAvian (EUNA1-2) and another NA of Gent1984 (EUNA2-7) or triple-gene RNA particles encoding one NA antigen each of EurAsianAvian (EUNA1-2), Gent1984 (EUNA2-7) and Scot1994 (EUHNA2-6) or Pdm09 (EUNA1-4) lineage IAV-S strains in different positions.

FIG. 13A-13D show results of evaluation of vaccine efficacy of a multivalent IAV-S vaccine, as described in Example 6. FIG. 13A: Induction of functional HI titers against heterologous IAV-S strains belonging to all four lineages; FIG. 13B: Induction of NI titers against homologous NA antigen of all three lineages; FIG. 13C: Rectal temperatures in pigs after experimental infection; and FIG. 13D: Lung lesions induced in pigs after experimental infection.

FIG. 14A-14B: Results of evaluation of vaccine efficacy after intradermal administration of a multivalent IAV-S vaccine, as described in Example 7. FIG. 14A: Induction of functional HI titers against heterologous IAV-S strains belonging to three out of four lineages tested. Induction of NI titers against two out of three homologous NA antigens tested.

### DEFINITION OF TERMS

In order to fully appreciate the invention, the following definitions are provided.

A nucleic acid construct is an artificially constructed segment of nucleic acid (e.g. DNA, RNA, mRNA), typically for transplantation into a target cell.

The use of singular terms for convenience in description is in no way intended to be limiting. Thus, for example, reference to a composition comprising “a polypeptide” includes reference to one or more of such polypeptides. In addition, reference to an “alphavirus RNA replicon particle” includes reference to a plurality of such alphavirus RNA replicon particles, unless otherwise indicated.

As used herein the term “approximately” is used interchangeably with the term “about” and signifies that a value is within fifty percent of the indicated value i.e., a composition containing “approximately” 1×10⁸ alphavirus RNA replicon particles per milliliter contains from 5×10⁷ to 1.5×10⁸ alphavirus RNA replicon particles per milliliter.

As used herein, the term “pig” or “swine” or “porcine” are used interchangeably and include all domesticated porcine species, unless otherwise indicated.

As used herein, a “phylogenetic cluster” is a set of influenza virus antigens, such as hemagglutinins (HAs) or neuraminidases (NAs), that have been grouped together (on the same branch) in a phylogenetic tree or evolutionary tree that is rooted back to a similar (homologous) ancestor. For the IAV-S neuraminidases and hemagglutinins found in the U.S., the predominant phylogenetic clusters are described in: [Anderson et al., Influenza and other Respiratory Viruses 7 (Suppl. 4): 42-51 (2013)].

As used herein, a “lineage” is a set of influenza virus hemagglutinins that have been grouped together (on the same branch) in an evolutionary tree that is rooted back to a similar (homologous) ancestor. These groupings have been made for European hemagglutinins and neuraminidase and are analogous to the phylogenetic clusters for U.S. viruses, but are not equivalent. Lineage determinations can be obtained by phylogenetic analysis of HA or NA sequences in question with pre-established reference sequences using readily available software, i.e., Clustal Omega [Sievers F., et al., (2011) Mol. Syst. Biol. 7:539] or a web-accessible annotation tool for H1 HA sequences [Anderson T K, et al., mSphere, 2016; 1 (6): e00275-16].

For the IAV-S hemagglutinin (HAs) found in Europe, there are four predominant lineages, as described in: [Watson et al., J. Virol. 89:9920-9931 (2015)] which correspond to 3 H1 HA clades described in Anderson et al., mSphere 1 (6): e00275-16 (2016) and one H3 HA clade [Anderson et al., unpublished]. European swine were infected solely by CS lineage viruses until 1979, when an avian H1N1 virus called “Eurasian avian-like swine H1N1” (EA), genetically distinct from the CS lineage, was isolated from pigs in Belgium and Germany. The EA lineage continues to circulate among European swine and has reassorted with human seasonal origin viruses since its emergence, resulting in the cocirculation of three distinct virus subtypes in Europe: (i) Eurasian avian-like H1ₐᵥN1 (EA or clade 1C.2.); (ii) A/swine/Gent/1/1984-like H3N2 (Gent/84 or clade 3.1970.1); and (iii) A/swine/Scotland/410440/1994-like H1huN2 (Scot/94 or clade 1B.1). Since April 2009, a novel H1N1 IAV virus, named (iv) A (H1N1) pdm09 or clade 1A.3.3.2 of swine origin spreads throughout the human population. In the context of the present invention, these four lineages are thus referred to as “EA”, “Gent/84”, “Scot/94” and “pdm09”.

As used herein, the term “replicon” refers to a modified RNA viral genome that lacks one or more elements (e.g., coding sequences for structural proteins) that if they were present, would enable the successful propagation of the parental virus in cell cultures or animal hosts. In suitable cellular contexts, the replicon will amplify itself and may produce one or more sub-genomic RNA species.

As used herein, the term “RNA replicon particle”, abbreviated “RP”, is an RNA replicon packaged in structural proteins, e.g., the capsid and glycoproteins, which may be derived from an alphavirus, e.g., is an alphavirus RNA replicon particle as described by Pushko et al., [Virology 239 (2): 389-401 (1997)], but may also be a Sindbis virus [Bredenbeek et al., 1993, J. of Virol., vol. 67, p. 6439-6446], and Semliki Forest virus [Liljestrom & Garoff, 1991, Biotechnology (NY), vol. 9, p. 1356-1361]. An RP cannot propagate in cell cultures or animal hosts (without a helper plasmid or analogous component), because the replicon does not encode the alphavirus structural components (e.g., capsid and glycoproteins). Preferably, the RNA RP of the present invention is an alphavirus RNA RP.

The term “non-IAV-S”, is used to modify terms such as pathogen, and/or antigen (or immunogen) to signify that the respective pathogen, and/or antigen (or immunogen) is neither an IAV-S pathogen nor an IAV-S antigen (or immunogen) and that a non-IAV-S protein antigen (or immunogen) does not originate from an IAV-S.

The term “originate(s) from” is used herein to signify that the unmodified and/or truncated amino acid sequence of that given protein antigen is encoded by that pathogen or strain of that pathogen. The coding sequence, within a nucleic acid construct of the present invention for a protein antigen originating from a pathogen may have been genetically manipulated so as to result in a modification and/or truncation of the amino acid sequence of the expressed protein antigen relative to the corresponding sequence of that protein antigen in the pathogen or strain of pathogen (including naturally attenuated strains) it originates from.

As used herein, the terms “treatment” or “treating”, “prevention” or “preventing”, “protecting”, or “providing protection to”, or “eliciting protective immunity to”, “aids in prevention of disease”, and “aids in the protection” do not require complete protection from any indication of infection. For example, “for use in the prevention” ” can mean that the provided protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection. Hence, the terms “prevention of a disease” or “treatment” encompass a prophylactic treatment against infection with the virus or against a disorder arising from the infection.

As used herein, a “vaccine” is a composition that is suitable for application to an animal, e.g., porcine (including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a disease arising from an infection with a wild-type micro-organism, i.e., strong enough for aiding in the prevention of the disease, and/or preventing, ameliorating or curing the disease.

As used herein, a multivalent vaccine is a vaccine that comprises two or more different antigens. In a particular embodiment of this type, the multivalent vaccine stimulates the immune system of the recipient against two or more different pathogens.

The terms “adjuvant” and “immune stimulant” are used interchangeably herein, and are defined as one or more substances that cause stimulation of the immune system. In this context, an adjuvant is used to enhance an immune response to one or more vaccine antigens/isolates. Accordingly, “adjuvants” are agents that nonspecifically increase an immune response to a particular antigen, thus reducing the quantity of antigen necessary in any given vaccine, and/or the frequency of injection necessary in order to generate an adequate immune response to the antigen of interest. In this context, an adjuvant is used to enhance an immune response to one or more vaccine antigens/isolates.

As used herein, a “nonadjuvanted vaccine” is a vaccine or a multivalent vaccine that does not contain an adjuvant.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient animal, e.g., porcine.

“Parenteral administration” includes subcutaneous injections, submucosal injections, intravenous injections, intramuscular injections, intradermal injections, and infusion.

Hemagglutinin and neuraminidase antigens of IAV-S may relate to the complete, i.e. the full-length, protein as specified in the sequences as defined herein or may relate to an antigenic fragment thereof, which fragment may equally be suitable to induce an adequate immunological response as is commonly known in the art of influenza vaccines (see e.g. PLOS ONE Research Article “An Influenza A/H1N1/2009 Hemagglutinin Vaccine Produced in Escherichia coli”, José M. Aguilar-Yáñez et al. Jul. 22, 2010; doi.org/10.1371/journal.pone.0011694; Vaccines (Basel) “Optimal Use of Vaccines for Control of Influenza A Virus in Swine”, Matthew R. Sandbulte et al. 2015 Marc 3 (1) 22-73).

In general, an antigenic fragment of a particular protein (e.g., a protein antigen) is a fragment of that protein that is antigenic, i.e., capable of specifically interacting with an antigen recognition molecule of the immune system, such as an immunoglobulin (antibody) or T cell antigen receptor. For example, an antigenic fragment of an IAV-S hemagglutinin (HA) is a fragment of the HA protein that is antigenic, i.e. it fulfills the function of an immunogenic epitope. Preferably, an antigenic fragment of the present invention is immunodominant for antibody and/or T cell receptor recognition. In particular embodiments, an antigenic fragment with respect to a given protein antigen is a fragment of that protein that retains at least 25% of the antigenicity (i.e. capability of inducing the corresponding antibodies as established by an HI or NI inhibition assay as described below) of the full-length protein. In preferred embodiments an antigenic fragment retains at least 50% of the antigenicity of the full-length protein. In more preferred embodiments, an antigenic fragment retains at least 75% of the antigenicity of the full length protein. Antigenic fragments can be as small as 20 amino acids or at the other extreme, be large fragments that are missing as little as a single amino acid from the full-length protein. In particular embodiments the antigenic fragment comprises 25 to 150 amino acid residues. In other embodiments, the antigenic fragment comprises 50 to 250 amino acid residues.

As used herein one amino acid sequence is 100% “identical” or has 100% “sequence identity” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

As used herein, nucleotide and amino acid sequence percent identity can be determined using a web based Clustal Omega, a multiple sequence alignment program with default parameters [Sievers and Higgins, Protein Sci. 2018 January; 27 (1): 135-1452018]. The percent identity value is a single numeric score determined for each pair of aligned sequences. It measures the number of identical residues (“matches”) in relation to the length of the alignment. Next to Clustal Omega, other programs, which may be used to determine nucleotide and amino acid sequence percent identity, are C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. Alternatively, an Advanced Blast search under the default filter conditions can be used, e.g., using the GCG (Genetics Computer Group, Program Manual for the GCG Package, Version 7, Madison, Wisconsin) pileup program using the default parameters.

### DETAILED DESCRIPTION OF THE INVENTION

The first embodiment of the first aspect of the present invention relates to a first nucleic acid construct, which combines at least first and second nucleic acid sequences encoding hemagglutinin (HA) antigens in a specific order. The first HA antigen encoded by the first nucleic acid sequence in direction from 5′ to 3′ of the nucleic acid construct is of the Scot/94 lineage. The second HA antigen encoded by the second nucleic acid sequence in direction from 5′ to 3′ of the nucleic acid construct is of the EA lineage.

The first HA antigen of the Scot/94 lineage may be of any strain, such as from strain A/swine/Italy/3033-1/2015 (H1N2) or A/swine/France/35-140041 (H1N2). In a preferred embodiment the first HA antigen of the Scot/94 lineage is from strain A/swine/Italy/3033-1/2015 (H1N2).

Further preferably, the first HA antigen comprises, and further more preferably consists of the amino acid sequence according to SEQ ID NO: 3 or an amino acid sequence having at least 85%, at least 87%, at least 89%, at least 90%, 91%, 92%, 93%, 94%, 95%, 96%, 97%, 98%, or 99% sequence identity. Further preferably, the first HA antigen consists of the amino acid sequence of SEQ ID NO: 3 or an amino acid sequence having at least 90%, preferably at least 93%, more preferably at least 95%, 96%, 97%, 98%, or 99% sequence identity.

The second HA antigen of the EA lineage may be of any strain, such as from strain A/swine/Denmark/101048-2/2011 (H1N1), A/swine/Italy/28762-3/2013 (H1N1) or A/swine/France/44-120070/2012 (H1N1). In a preferred embodiment the second HA antigen of the EA lineage is from strain A/swine/Italy/28762-3/2013 (H1N1).

Further preferably, the second HA antigen comprises, and further more preferably consists of the amino acid sequence according to SEQ ID NO: 6 or an amino acid sequence having at least at least 90%, 91%, 92%, 93%, 94%, 95%, 96%, 97%, 98%, or 99% sequence identity. Further preferably, the second HA antigen consists of the amino acid sequence of SEQ ID NO: 6 or an amino acid sequence having at least 93%, preferably at least 95%, further preferably at least 96%, 97%, 98%, or 99% sequence identity.

The second embodiment of the present invention relates to a second nucleic acid construct, which combines at least first and second nucleic acid sequences encoding hemagglutinin (HA) antigens in a specific order. The first HA antigen encoded by the first nucleic acid sequence in direction from 5′ to 3′ of the nucleic acid construct is of the Gent/84 lineage. The second HA antigen encoded by the second nucleic acid sequence in direction from 5′ to 3′ of the nucleic acid construct is of the pdm09 lineage.

The first HA antigen of the Gent/84 lineage may be of any strain, such as from strain A/swine/Italy/240849/2015 (H3N2). In a preferred embodiment the first HA antigen of the Gent/84 lineage is from strain A/swine/Italy/240849/2015 (H3N2).

Further preferably, the first HA antigen comprises, and further more preferably consists of the amino acid sequence according to SEQ ID NO: 9 or an amino acid sequence having at least at least 90%, 91%, 92%, 93%, 94%, 95%, 96%, 97%, 98%, or 99% sequence identity. Further preferably, the first HA antigen consists of the amino acid sequence of SEQ ID NO: 9 or an amino acid sequence having at least 93%, preferably at least 95%, further preferably at least 96%, 97%, 98%, or 99% sequence identity.

The second HA antigen of the pdm09 lineage may be of any strain, such as from strain A/swine/England/373/2010 (H1N1). In a preferred embodiment the second HA of the EA lineage is from strain A/swine/England/373/2010 (H1N1).

Further preferably, the second HA antigen comprises, and further more preferably consists of the amino acid sequence according to SEQ ID NO: 12 or an amino acid sequence having at least at least 95%, 96%, 97%, 98%, or 99% sequence identity. Further preferably, the first HA antigen consists of the amino acid sequence of SEQ ID NO: 12 or an amino acid sequence having at least 95%, preferably at least 96%, further preferably at least 97%, 98%, or 99% sequence identity.

In the first embodiment of the second aspect, there is provided a nucleic acid construct comprising first and second nucleic acid sequences:
a first nucleic acid sequence encoding a first HA antigen of IAV-S of the A/swine/Scotland/410440/1994-like H1ₕᵤN2 (Scot/94) lineage from strain A/swine/Italy/3033-1/2015 (H1N2), and
a second nucleic acid sequence encoding a second HA antigen of IAV-S of the Eurasian avian-like H1ₐᵥN1 (EA) lineage from strain A/swine/Italy/28762-3/2013 (H1N1).

Preferably, the amino acid sequence of the first HA antigen of IAV-S of the Scot/94 lineage from strain A/swine/Italy/3033-1/2015 (H1N2) comprises, and further preferably consists of a sequence of SEQ ID NO: 3 or an amino acid sequence having at least 85%, preferably at least 90% sequence identity. The amino acid identity is further preferably at least 91%, 92%, more preferably at least 93%, 94%, 95%, 96%, 97%, 98% or even 99% or more.

Preferably, the amino acid sequence of the second HA antigen of IAV-S of the EA lineage from strain A/swine/Italy/28762-3/2013 (H1N1) comprises, and further preferably consists of a sequence of SEQ ID NO: 6 or an amino acid sequence having at least 90%, preferably at least 93% sequence identity. The amino acid identity is further preferably at least 94%, 95%, more preferably at least 96%, 97%, 98% or even 99% or more.

In the second embodiment of the second aspect, there is provided a nucleic acid construct for use in the prevention or treatment of a disease caused by a Swine influenza A virus in a subject, the nucleic acid construct comprising first and second nucleic acid sequences:
the first nucleic acid sequence encoding a first hemagglutinin (HA) antigen of a Swine influenza A virus (IAV-S) of the A/swine/Gent/1/1984-like H3N2 (Gent/84) lineage from strain A/swine/Italy/240849/2015 (H3N2), and
a second nucleic acid sequence encoding a second HA antigen of IAV-S of the A (H1N1) pdm09 (pdm09) lineage from strain A/swine/England/373/2010 (H1N1).

Preferably, the amino acid sequence of the first HA antigen of IAV-S of the Gent/84 lineage from strain A/swine/Italy/240849/2015 (H3N2) comprises, and further preferably consists of a sequence of SEQ ID NO: 9 or an amino acid sequence having at least 90%, preferably at least 95% sequence identity. The amino acid identity is preferably at least 96%, 97%, more preferably at least 98% or even 99% or more.

Preferably, the amino acid sequence of the second HA antigen of IAV-S of the pdm09 lineage from strain A/swine/England/373/2010 (H1N1) comprises, and further preferably consists of a sequence of SEQ ID NO: 12 or an amino acid sequence having at least 90%, preferably at least 95% sequence identity. The amino acid identity is preferably at least 96%, 97%, more preferably at least 98% or even 99% or more.

The nucleic acid constructs according to the first and/or second embodiment of the first and/or second aspect may be included in an expression cassette incorporating the nucleic acid sequences encoding hemagglutinin (HA) antigens as described above as heterologous genes together with transcription—and/or expression controlling nucleic acid sequences, such as alphavirus sub-genomic promoter sequences etc, and which are suitable for expression of the HA antigens. Such expression cassettes can be generated using well known techniques by incorporating the heterologous nucleic acid sequences encoding the HA antigens in a vector, such as DNA vectors or RNA vectors. The vector can be a viral replicon backbone, such as an RNA replicon particle (RP), and preferably is an alphavirus RNA replicon particle.

Thus, in the first and second aspect of the present invention, there is further provided an RNA RP, preferably an alphavirus RNA RP, comprising the nucleotide construct according to the first embodiment. In addition, the present invention further provides an RNA preferably an alphavirus RNA RP, comprising the nucleotide construct according to the second embodiment.

An “alphavirus RNA replicon particle (RP)” is well known as “non-transmissible”, “single-cycle”, or “propagation-incompetent” virus like particle vector. The genome can encode one or more heterologous genes from its 26S subgenomic promoter. The RP can replicate within the target cell without producing a progeny, and in this way deliver and express heterologous antigen(s) to the immune system of a target animal. The alphavirus RNA RP may be based on a human Venezuelan equine encephalitis vaccine (VEEV) TC-83 strain.

RP expression systems for heterologous expression of antigens are available in the art, and include, for example, the commercially available RP vector-based platforms for the manufacture of vaccines, such as the Alphavaccine Platform System based on the VEE virus and the SEQUIVITY™ Technology available from MSD/Merck Animal Health, USA. Thus, in a further preferred embodiment, the RNA replicon particle is a Venezuelan Equine Encephalitis (VEE) alphavirus-based RNA replicon particle.

For example, the viral HA antigen gene(s) can then be expressed from the (26S-Alphavirus) subgenomic promoter, and transcribed replicon RNA can be packaged into RPs by expression of the structural proteins by a packaging cell lines, or via co-transfection into suitable host cells of the replicon RNA and of one or more ‘helper’ RNA's encoding the structural proteins. The generation of VEE TC-83 RNA replicon particles is for example described in U.S. Pat. Nos. 9,441,247 and 8,460,913. In short, the HA or NA genes were de novo synthesized (DNA2.0) using sequence from SIV strains. Two HA or three NA genes were cloned into a replicon vector plasmid using monodirectional expression cassettes in tandem and the sequence was confirmed to ensure no mutations were introduced in the cloning process. RNA was generated by in vitro transcription of linearized replicon plasmid DNA using T7 RNA polymerase as described previously [Kamrud et al., Virology. 2007; 360 (2): 376-387]. RP were generated by co-electroporation of HA or NA replicon RNA and structural gene helper RNAs into Vero cells and subsequent harvest of the particles [Hooper et al., Vaccine. 2009; 28 (2): 494-511].

General molecular biological techniques involving cloning, transfection, recombination, selection, and amplification are for example explained in great detail in standard text-books like Sambrook & Russell: “Molecular cloning: a laboratory manual” [2001, Cold Spring Harbour Laboratory Press; ISBN: 0879695773; Ausubel et al., in: Current Protocols in Molecular Biology, J. Wiley and Sons Inc., NY, 2003, ISBN: 047150338X; C. Dieffenbach & G. Dveksler: “PCR primers: a laboratory manual”, CSHL Press, ISBN 0879696540; and “PCR protocols”, by: J. Bartlett and D. Stirling, Humana press, ISBN: 0896036421].

The nucleic acid constructs of the present invention can be used in immunogenic compositions comprising the nucleic acid constructs. Preferably, the immunogenic compositions comprise one of more replicon particles comprising the nucleic acid constructs of the present invention. Thus, the replicon particles of the present invention can be used in immunogenic compositions, such as vaccines, comprising the replicon particles. The immunogenic compositions or vaccines may consist of the replicon particles or may comprise the replicon particles in combination with additional components, such as carriers or adjuvants. The immunogenic compositions of the present invention may be used in vaccines for use in the prevention of a disease caused by a Swine influenza A virus (IAV-S) in a subject.

Thus, in the first and/or second aspects, the present invention further provides an immunogenic composition comprising or consisting of RNA RP comprising the nucleic acid construct according to the first embodiment. Alternatively, the present invention further provides an immunogenic composition comprising or consisting of RNA RP comprising the nucleic acid construct according to the second embodiment.

In a preferred embodiment of the first and/or second aspects, the present invention provides an immunogenic composition comprising a first RNA RP comprising the nucleotide construct according to the first embodiment and a second RNA RP comprising the nucleotide construct according to the second embodiment. It could be shown in the present invention that an immunogenic composition comprising a combination of replicon particles according to the first and second embodiment provide for broad protection against the existing IAV-S lineages, and thus such an immunogenic composition can beneficially be used as a vaccine that aid in the protection, i.e. that aid in the prevention or treatment, of the vaccinated subject, such as swine (e.g. sow or piglet) against IAV-S infection.

Therefore, in a preferred embodiment, the present invention provides an immunogenic composition, such as vaccine, comprising first and second RNA replicon particles,
(i) the first RNA replicon particle, preferably an alphavirus RNA replicon particle, comprising a nucleic acid construct, comprising first and second nucleic acid sequences encoding first and second hemagglutinin (HA) antigens of a Swine influenza A virus (IAV-S), wherein
   the first HA antigen is a of the Gent/84 lineage, and
   the second HA antigen is of the pdm09 lineage,
(ii) the second RNA replicon particle, preferably an alphavirus RNA replicon particle, comprising a nucleic acid construct comprising third and fourth nucleic acid sequences encoding third and fourth HA antigens of IAV-S, wherein
   the third HA antigen is of the Scot/94 lineage, and
   the fourth HA antigen is of the EA lineage.

In a particularly preferred embodiment, the present invention provides an immunogenic composition, such as a vaccine, that comprises first and second RNA replicon particles:
(i) the first RNA replicon particle, preferably an alphavirus RNA replicon particle,
comprising a first nucleic acid construct comprising, in the order from 5′ to 3′ of the nucleic acid sequence:
a first nucleic acid sequence encoding a first HA antigen of IAV-S of the Scot/94 lineage, and
a second nucleic acid sequence encoding a second HA antigen of IAV-S of the EA lineage, and the immunogenic composition further comprises:
(ii) the second RNA replicon particle, preferably an alphavirus RNA replicon particle, comprising a second nucleic acid construct comprising, in the order from 5′ to 3′ of the nucleic acid sequence:
a third nucleic acid sequence encoding a third HA antigen of IAV-S of the Gent/84 lineage, and
a fourth nucleic acid sequence encoding a fourth HA antigen of IAV-S of the pdm09 lineage.

In a particularly preferred embodiment, the present invention provides an immunogenic composition, such as a vaccine, that comprises first and second RNA replicon particles:
(i) the first RNA replicon particle, preferably an alphavirus RNA replicon particle, comprising a first nucleic acid construct comprising, in the order from 5′ to 3′ of the nucleic acid sequence:
a first nucleic acid sequence encoding a first HA antigen of IAV-S of the Scot/94 lineage, and
a second nucleic acid sequence encoding a second HA antigen of IAV-S of the EA lineage, and the immunogenic composition further comprises:
(ii) the second RNA replicon particle, preferably an alphavirus RNA replicon particle, comprising a second nucleic acid construct comprising, in the order from 5′ to 3′ of the nucleic acid sequence:
a third nucleic acid sequence encoding a third HA antigen of IAV-S of the Gent/84 lineage, and
a fourth nucleic acid sequence encoding a fourth HA antigen of IAV-S of the pdm09 lineage.

Thus, in a third aspect the present invention provides an immunogenic composition, such as vaccine, comprising first and second RNA replicon particles,
(i) the first RNA replicon particle, preferably an alphavirus RNA replicon particle, comprising a nucleic acid construct, comprising first and second nucleic acid sequences encoding, in the order from 5′ to 3′ of the nucleic acid sequence, first and second hemagglutinin (HA) antigens of a Swine influenza A virus (IAV-S), wherein
   the first HA antigen encoded by the first nucleic acid sequence is of the Gent/84 lineage from strain A/swine/Italy/240849/2015 (H3N2), preferably of SEQ ID NO. 9 or an amino acid sequence having at least 90% sequence identity thereof, and
   the second HA antigen encoded by the first nucleic acid sequence is of the pdm09 lineage from strain A/swine/England/373/2010 (H1N1), preferably of SEQ ID NO. 12 or an amino acid sequence having at least 95% sequence identity thereof
(ii) the second RNA replicon particle, preferably an alphavirus RNA replicon particle, comprising a nucleic acid construct comprising third and fourth nucleic acid sequences encoding, in the order from 5′ to 3′ of the nucleic acid sequence, third and fourth HA antigens of IAV-S, wherein
   the third HA antigen encoded by the third nucleic acid sequence is of the Scot/94 lineage from strain A/swine/Italy/3033-1/2015 (H1N2), preferably of SEQ ID NO. 3, or an amino acid sequence having at least 85% sequence identity thereof, and
   the fourth HA antigen encoded by the fourth nucleic acid sequence is of the EA lineage from strain A/swine/Italy/28762-3/2013 (H1N1), preferably of SEQ ID NO. 6, or an amino acid sequence having at least 90% sequence identity thereof.

The nucleic acid constructs, immunogenic compositions and replicon particles of the third aspects are as described above for the first and second aspects of the present invention. Thus, further encompassed by the present invention are any combinations of the embodiments of the third aspect with the embodiments of the first and second aspects as described herein. Thus, the present invention further provides replicon particles as described in the third aspect, wherein the nucleic acid constructs encode IAV-S HA antigens, which are arranged in the specific order as defined in the first aspect and/or in which the IAV-S antigens are from the specific strains as defined in the second aspect.

The immunogenic composition may be adapted for simultaneous or consecutive administration of the first and second RNA replicon particles as described above, i.e. for simultaneous or consecutive administration of the RNA RP comprising the nucleic acid constructs according to the first and second embodiment. Preferably, the immunogenic composition is adapted for simultaneous administration of the first and second RNA replicon particles. Hence, in the preferred embodiment, the immunogenic composition comprises the first and second RNA replicon particles in a unit dosage form.

In a further preferred embodiment, the immunogenic composition may comprise one or more additional RNA replicon particles. Such additional RNA RPs may comprise nucleic acid constructs encoding one or more additional antigens. For example, the additional RNA RPs may comprise nucleic acid constructs encoding one or more neuraminidase (NA) antigens of IAV-S. In specific embodiments, the nucleic acid constructs encode two or three, preferably three NA antigens of IAV-S, or immunogenic fragments thereof.

In a particularly preferred embodiment, the additional RNA RPs comprises a nucleic acid construct comprising first, second and third nucleic acid sequences encoding first, second and third NA antigens of IAV-S, wherein
the first NA antigen is of the Scot/94 lineage,
the second NA antigen is of the Gent/84 lineage, and
the third NA antigen is selected from the pdm09 lineage or the EA lineage.

Accordingly, in a fourth aspect of the present invention, there is provided a nucleic acid construct comprising first, second and third nucleic acid sequences encoding first, second and third NA antigens of IAV-S, wherein
the first NA antigen encoded by the first nucleic acid sequence is of the Scot/94 lineage,
the second NA antigen encoded by the second nucleic acid sequence is of the Gent/84 lineage, and
   the third NA antigen encoded by the third nucleic acid sequence is selected from the pdm09 lineage or the EA lineage.

Preferably, the amino acid sequence of the first NA antigen of IAV-S of the Scot/94 lineage is from strain A/swine/England/61470/2013 (H1N2). The amino acid sequence of the first NA antigen preferably comprises, and further preferably consists of a sequence of SEQ ID NO: 15 or an amino acid sequence having at least 90% sequence identity. The amino acid identity is preferably at least 96%, 97%, more preferably at least 98% or even 99% or more.

Preferably, the amino acid sequence of the second NA antigen of IAV-S of the Gent/84 lineage is from strain A/swine/Italy/248147-8/2015 (H3N2). The amino acid sequence of the second NA antigen preferably comprises, and further preferably consists of a sequence of SEQ ID NO: 18 or an amino acid sequence having at least 90% sequence identity. The amino acid identity is preferably at least 96%, 97%, more preferably at least 98% or even 99% or more.

Preferably, the amino acid sequence of the third NA antigen of IAV-S of the pdm09 lineage is from strain A/swine/England/373/2010 (H1N1) or A/swine/Italy/179057/2015 (H1N1), preferably from strain A/swine/Italy/179057/2015 (H1N1). The amino acid sequence of the third NA antigen preferably comprises, and further preferably consists of a sequence of SEQ ID NO: 21 or an amino acid sequence having at least 90% sequence identity. The amino acid identity is preferably at least 96%, 97%, more preferably at least 98% or even 99% or more.

Alternatively, the amino acid sequence of the third NA antigen of IAV-S of the EA lineage is from strain A/swine/Italy/28762-3/2013 (H1N1). The amino acid sequence of the third NA antigen preferably comprises, and further preferably consists of a sequence of SEQ ID NO: 24 or an amino acid sequence having at least 90% sequence identity. The amino acid identity is preferably at least 96%, 97%, more preferably at least 98% or even 99% or more.

There is further provided an RNA replicon particle, preferably an alphavirus RNA replicon particle, comprising a nucleic acid construct comprising first, second and third nucleic acid sequences encoding first, second and third neuraminidase (NA) antigens of a Swine influenza A virus (IAV-S), wherein
the first NA antigen is of the Scot/94 lineage,
the second NA antigen is of the Gent/84 lineage, and
the third NA antigen is selected from the pdm09 lineage or the EA lineage.

The replicon particle comprising the nucleic acid construct according to the fourth aspect may be used alone or in combination with the replicon particle according to the first, second and/or third aspect of the present invention as described herein and is beneficially be used in combination with the replicon particle comprising the hemagglutinin antigens as described in the first, second and/or third aspects of the present invention.

The replicon particle according to this fourth aspect is not particularly limited and is preferably a replicon particle, such as an alphavirus replicon particle, most preferably a Venezuelan Equine Encephalitis Virus (VEEV) alphavirus RNA replicon particle as described in the first, second and/or third aspect.

In a further preferred embodiment, the present invention provides an immunogenic composition, such as a vaccine, comprising at least first, second and third RNA replicon particles,
the first RNA replicon particle comprising a nucleic acid construct, comprising, in the order from 5′ to 3′ of the nucleic acid sequence, first and second nucleic acid sequences encoding first and second HA antigens of IAV-S, wherein
   the first HA antigen is of the Scot/94 lineage, and
   the second HA antigen is of the EA lineage,
the second RNA replicon particle comprising a nucleic acid construct comprising, in the order from 5′ to 3′ of the nucleic acid sequence, third and fourth nucleic acid sequences encoding third and fourth HA antigens of IAV-S, wherein
   the third HA antigen is of the Gent/84 lineage, and
   the fourth HA antigen is of the pdm09 lineage, and
the third RNA replicon particle comprises a nucleic acid construct comprising first, second and third nucleic acid sequences encoding first, second and third NA antigens of IAV-S, wherein
   the first NA antigen is of the Scot/94 lineage,
   the second NA antigen is of the Gent/84 lineage, and
   the third NA antigen is selected from the pdm09 lineage or the EA lineage

The immunogenic composition described above, such as a vaccine, can beneficially be used as a vaccine that aid in the protection of the vaccinated subject, such as swine (e.g. sow or piglet) against IAV-S infection.

The immunogenic composition may be adapted for simultaneous or consecutive administration of the first, second and third RNA replicon particles as described above, i.e. for simultaneous or consecutive administration of the RNA RP comprising the nucleic acid constructs according to the first, second and/or third aspect in combination with the nucleic acid construct according to the fourth aspect. Preferably, the immunogenic composition is adapted for simultaneous administration of the first, second and third RNA replicon particles. Hence, in the preferred embodiment, the immunogenic composition comprises the first, second and third RNA replicon particles in a unit dosage form.

The present invention also provides vaccines against multiple porcine pathogens. For example, the coding sequence of a protein antigen or antigenic fragment thereof, or combination of such coding sequences of protein antigens useful in a porcine vaccine can be added to an RNA replicon particle (RP) and/or combined in the same RP as one that encodes an HA or NA originating from an IAV-S in the vaccine, as described herein. Examples of pathogens that one or more of protein antigens or antigenic fragments thereof can originate from include porcine reproductive and respiratory syndrome virus (PRRS), porcine circovirus (PCV), transmissible gastroenteritis virus (TGE), porcine pseudorabies virus (PPRV), porcine parvovirus (PPV), porcine rotavirus (PRV), porcine epidemic diarrhea virus (PED), Pasteurella multocida of multiple serotypes, Salmonella ssp., Escherichia coli, e.g., (serotypes K99, K88, 987P, or F41), Haemophilus parasuis, Lawsonia intracellularis, Mycoplasma ssp. (e.g., Mycoplasma hyopneumoniae), Bordetella bronchiseptica, Erysipelas ssp., Campylobacter ssp., Actinobacillus pleuropneumoniae, Clostridium perfringens, and Clostridium difficile.

In addition, the present invention provides vaccines comprising one or more RPs of the present invention in combination with one or more other vectors encoding one or more of these porcine antigens (e.g., a baculovirus vector encoding an ORF-2 protein from a porcine circovirus-2, (PCV-2) and/or porcine circovirus-3 (PCV-3) and/or inactivated toxoids originating from one or more of these porcine pathogens. Moreover, such vaccines can include any RNA replicon particle that encodes a HA and/or NA originating from an IAV-S in a vaccine of the present invention together with one or more killed and/or modified (attenuated) live porcine virus isolates and/or porcine bacteria.

Accordingly, one or more RNA RPs that encode one or more HAs and/or NAs originating from IAV-S can be added together with one or more other vectors encoding one or more porcine antigen and/or one or more killed and/or modified (attenuated) live virus isolates such as one or more killed or modified live IAS-V strain, one or more killed and/or modified live PRRS virus, one or more killed and/or modified live PCV, one or more killed, and/or modified live TGE, one or more killed and/or modified live PPRV, one or more killed and/or modified live PPV, one or more killed and/or modified live PRV and one or more killed and/or modified live PED. Moreover, one or more alphavirus RNA replicon particles (RPs) that encode one or more HAs or NAs originating from IAV-S can be added together with one or more other vectors encoding one or more porcine antigen and/or added together with one or more killed and/or modified (attenuated) live bacteria that can infect swine too, including one or more killed and/or modified live Pasteurella multocida (of one or more multiple serotypes), Salmonella ssp., Escherichia coli (of one or more multiple serotypes), Haemophilus parasuis, Lawsonia intracellularis, Mycoplasma ssp., (e.g., Mycoplasma hyopneumoniae), Bordetella bronchiseptica, Erysipelas ssp., Campylobacter ssp., Actinobacillus pleuropneumoniae, Clostridium perfringens, and Clostridium difficile.

Accordingly, the present invention also includes all of the RNA replicon particles of the present invention, naked DNA vectors that comprise the nucleic acid constructs of the present invention, naked RNA vectors that comprise the nucleic acid constructs of the present invention, the nucleic acid constructs of the present invention including synthetic messenger RNA, and RNA replicons, as well as all of the immunogenic compositions and/or vaccines that comprise the nucleic acid constructs (e.g., synthetic messenger RNA, RNA replicons), the alphavirus RNA replicon particles, naked RNA vectors, and/or the naked DNA vectors of the present invention.

The immunogenic composition of the present invention can be used as a vaccine, which may be a non-adjuvanted vaccine or an adjuvanted vaccine. Thus, the present invention further comprises vaccines (multivalent) vaccines comprising the immunogenic compositions of the present invention. In particular embodiments, the vaccines are a nonadjuvanted vaccine. In other embodiments, the vaccines comprise an adjuvant. Adjuvants suitable for use in the vaccine of the present invention are not particularly limited, and may comprise one or more adjuvants selected from the group consisting of a biodegradable oil, an oil-in-water emulsion with 2.5-50% (v/v) mineral oil, and a biodegradable oil mixed with an oil-in-water emulsion with 2.5-50% (v/v) mineral oil.

In particular embodiments, the adjuvant is a biodegradable oil. In specific embodiments of this type, the biodegradable oil is dl-α-tocopheryl acetate (vitamin E acetate). In other embodiments, the adjuvant comprises an oil-in-water emulsion with 2.5%-50% (v/v) mineral oil. In specific embodiments the adjuvant comprises an oil-in-water emulsion with 2.5% (v/v) mineral oil. In related embodiments, the adjuvant comprises is an oil-in-water emulsion with 5% (v/v) mineral oil. In other embodiments, the adjuvant comprises an oil-in-water emulsion with 12.5% (v/v) mineral oil. In still other embodiments, the adjuvant comprises an oil-in-water emulsion with 25% (v/v) mineral oil. In yet other embodiments, the adjuvant comprises an oil-in-water emulsion with 50% (v/v) mineral oil. In more specific embodiments the adjuvant comprises a mixture of a biodegradable oil with a mineral oil adjuvant. In specific embodiments, the biodegradable oil is dl-α-tocopheryl acetate and the mineral oil is a liquid paraffin. In more specific embodiments, the biodegradable oil is dl-α-tocopheryl acetate and the mineral oil is a light liquid paraffin.

In related formulations, the adjuvant is a mixture of two components. The first component consists of mineral oil droplets with an approximate average (volume weighed) size around 1 μm, which is stabilized with polysorbate 80 (polyoxyethylene (20) sorbitan monooleate) in water. The first component can comprise 25 weight percent of the mineral oil and 1 weight percent of the polysorbate 80, with the remainder water. The second component can consist of droplets of biodegradable dl-α-tocopheryl acetate with an approximate average (volume weighed) size of 400 nm, which is also stabilized with polysorbate 80. Particular formulations comprise 15 weight percent of dl-α-tocopheryl acetate and 6 weight percent of polysorbate 80, with the remainder water. In particular embodiments, the adjuvant is X-SOLVE™ (which is a combination of two component adjuvants: DILUVAC FORTE™ which is based on dl-α-tocopheryl acetate and MICROSOL™, which is based on light liquid paraffin [see e.g., U.S. Pat. No. 8,597,662]. In related formulations, the adjuvant contains oil droplets of sub-micrometer size and droplets of biodegradable oil, with the droplets of the biodegradable oil having an average size that differs from the average size of the droplets of mineral oil [see e.g., U.S. Pat. No. 9,084,768].

In certain embodiments, the vaccine aids in the prevention of disease due to IAV-S. In related embodiments, antibodies are induced in a porcine subject when the porcine is immunized with the vaccine. In certain embodiments, the porcine subject is a sow. In related embodiments, the vaccine provides protective maternal antibodies to progeny of the vaccinated sow. In other embodiments, the porcine subject is a piglet. In particular embodiments of this type, the vaccine is administered to a piglet as early as 3 days of age. In specific embodiments, the vaccine is administered as a booster vaccine. In certain embodiments, the vaccine is administered as a single dose vaccine. In specific embodiments of this type, the vaccine is administered as a booster vaccine. In yet other embodiments, the vaccine is administered as a multi-dose vaccine. In specific embodiments of this type, the vaccine is administered as a two-dose vaccine.

The present invention also provides methods of immunizing a porcine (e.g., a sow or a piglet) against a porcine pathogen, e.g., IAV-S, comprising administering to the porcine an immunologically effective amount of a vaccine or multivalent of the present invention. In particular embodiments, the vaccine is administered via intramuscular injection. In alternative embodiments, the vaccine is administered via subcutaneous injection. In other embodiments, the vaccine is administered via intravenous injection. In still other embodiments, the vaccine is administered via intradermal injection. In yet other embodiments, the vaccine is administered via oral administration. In still other embodiments, the vaccine is administered via intranasal administration. A preferred method is intradermal administration. Another preferred method is intramuscular administration

Accordingly, the vaccines and multivalent vaccines of the present invention can be administered as a primer vaccine and/or as a booster vaccine. In specific embodiments, a vaccine of the present invention is administered as a one-shot vaccine (one dose), without requiring subsequent administrations. In certain embodiments, in the case of the administration of both a primer vaccine and a booster vaccine, the primer vaccine and the booster vaccine can be administered by the identical route.

In certain embodiments of this type, the primer vaccine and the booster vaccine are both administered by intradermal injection. In other embodiments of this type, the primer vaccine and the booster vaccine are both administered by intramuscular injection. In alternative embodiments, in the case of the administration of both a primer vaccine and a booster vaccine, the administration of the primer vaccine can be performed by one route and the booster vaccine by another route. In certain embodiments of this type, the primer vaccine can be administered by intradermal injection and the booster vaccine can be administered orally. In related embodiments of this type, the primer vaccine can be administered by intramuscular injection and the booster vaccine can be administered orally. In other embodiments of this type, the primer vaccine can be administered by intramuscular injection and the booster vaccine can be administered by intradermal injection. In still other embodiments of this type, the primer vaccine can be administered by intradermal injection and the booster vaccine can be administered by intramuscular injection. The skilled artisan will appreciate that the vaccine composition is preferably formulated appropriately for each type of recipient animal and route of administration.

The present invention further provides a method of immunizing a porcine against IAV-S, the method comprising administering to the porcine an immunologically effective amount of the vaccine of the present invention. The method preferably comprises intradermal administration of the vaccine. The invention further provides for a method of immunizing a porcine (e.g., a sow or a piglet) against IAV-S comprising injecting the porcine with an immunologically effective amount of the above described inventive vaccines, so that the porcine produces appropriate IAV-S antibodies. In particular embodiments, the vaccines can include from about 1×10⁴ to about 1×10¹⁰ RPs or higher, for example. In more particular embodiments, the vaccines can include from about 1×10⁵ to about 1×10⁹ RPs. In even more particular embodiments, the vaccines can include from about 1×10⁶ to about 1×10⁸ RPs.

In particular embodiments, the vaccines of the present invention are administered in 0.05 mL to 3 mL doses. In more particular embodiments, the dose administered is 0.1 mL to 2 mL. In still more particular embodiments, the dose administered is 0.2 mL to 1.5 mL. In even more particular embodiments, the dose administered is 0.3 to 1.0 mL. In still more particular embodiments, the dose administered is 0.4 mL to 0.8 mL.

Thus, in a first aspect, the present invention provides the following embodiments:
[1] A nucleic acid construct for use in the prevention of a disease caused by a Swine influenza A virus (IAV-S) in a subject, the nucleic acid construct comprising, in the order from 5′ to 3′ of the nucleic acid sequence:
   a first nucleic acid sequence encoding a first hemagglutinin (HA) antigen of IAV-S of the A/swine/Scotland/410440/1994-like H1ₕᵤN2 (Scot/94) lineage, and
   a second nucleic acid sequence encoding a second HA antigen of IAV-S of the Eurasian avian-like H1ₐᵥN1 (EA) lineage.
[2] The nucleic acid construct for use as defined in [1], wherein the first HA antigen is from strain A/swine/Italy/3033-1/2015 (H1N2).
[3] The nucleic acid construct for use as defined in [1] or [2], wherein the first HA antigen encoded by the first nucleic acid sequence comprises an amino acid sequence of SEQ ID NO: 3 or an amino acid sequence having at least 85% sequence identity thereof.
[4] The nucleic acid construct for use as defined in any one of [1] to [3], wherein the second HA antigen is from strain A/swine/Italy/28762-3/2013 (H1N1).
[5] The nucleic acid construct for use as defined in any one of [1] to [4], wherein the second HA antigen encoded by the second nucleic acid sequence comprises an amino acid sequence of SEQ ID NO: 6 or an amino acid sequence having at least 90% sequence identity thereof.
[6] A nucleic acid construct for use in the prevention of a disease caused by a Swine influenza A virus (IAV-S) in a subject, the nucleic acid construct comprising, in the order from 5′ to 3′ of the nucleic acid sequence:
   a first nucleic acid sequence encoding a first HA antigen of IAV-S of the A/swine/Gent/1/1984-like H3N2 (Gent/84) lineage, and
   a second nucleic acid sequence encoding a second HA antigen of IAV-S of the A (H1N1) pdm09 (pdm09) lineage.
[7] The nucleic acid construct for use as defined in [6], wherein the first HA antigen is from strain A/swine/Italy/240849/2015 (H3N2).
[8] The nucleic acid construct for use as defined in [6] or [7], wherein the first HA antigen encoded by the first nucleic acid sequence comprises an amino acid sequence of SEQ ID NO: 9 or an amino acid sequence having at least 90% sequence identity thereof.
[9] The nucleic acid construct for use as defined in any one of [6] to [8], wherein the second HA antigen is from strain A/swine/England/373/2010 (H1N1).
[10] The nucleic acid construct for use as defined in any one of [6] to [9], wherein the second HA antigen encoded by the second nucleic acid sequence comprises an amino acid sequence of SEQ ID NO: 12 or an amino acid sequence having at least 95% sequence identity thereof.
[11] An RNA replicon particle comprising the nucleic acid construct as defined in any one of [1] to [5].
[12] An RNA replicon particle comprising the nucleic acid construct as defined in any one of [6] to [10].
[13] The RNA replicon particle as defined in or [16], which is an alphavirus RNA replicon particle.
[14] The RNA replicon particle as defined in [13], which is a Venezuelan Equine Encephalitis (VEE) alphavirus RNA replicon particle
[15] An immunogenic composition, comprising the RNA replicon particle of any one of [11] to [14].
[16] The immunogenic composition as defined in comprising the RNA replicon particle of and [12].
[17] The immunogenic composition as defined in [16], which is adapted for simultaneous administration of the alphavirus RNA replicon particles as defined in and [12].
[18] A vaccine comprising the immunogenic composition as defined in any one of to [17].
[19] The vaccine as defined in [18], which is a nonadjuvanted vaccine.
[20] The vaccine as defined in [18], which comprises an adjuvant selected from the group consisting of a biodegradable oil, an oil-in-water emulsion with 2.5-50% (v/v) mineral oil, and a biodegradable oil mixed with an oil-in-water emulsion with 2.5-50% (v/v) mineral oil.
[21] The vaccine as defined in any one of to for use in the prevention of a disease caused by a Swine influenza A virus in a subject.
[22] A method of immunizing a porcine against a swine influenza A virus, the method comprising administering to the porcine an immunologically effective amount of the vaccine of any one of to [20].
[23] A nucleic acid construct comprising, in the order from 5′ to 3′ of the nucleic acid sequence:
   a first nucleic acid sequence encoding a first HA antigen of a Swine influenza A virus (IAV-S) of the A/swine/Scotland/410440/1994-like H1ₕᵤN2 (Scot/94) lineage, and
   a second nucleic acid sequence encoding a second HA antigen of IAV-S of the Eurasian avian-like H1ₐᵥN1 (EA) lineage.
[24] A nucleic acid construct comprising, in the order from 5′ to 3′ of the nucleic acid sequence:
   a first nucleic acid sequence encoding a first HA antigen of IAV-S of the A/swine/Gent/1/1984-like H3N2 (Gent/84) lineage, and
a second nucleic acid sequence encoding a second HA antigen of IAV-S of the A (H1N1) pdm09 (pdm09) lineage.

In a second aspect, the present invention provides the following embodiments:
[1] A nucleic acid construct for use in the prevention of a disease caused by a Swine influenza A virus in a subject, the nucleic acid construct comprising first and second nucleic acid sequences:
   a first nucleic acid sequence encoding a first HA antigen of IAV-S of the A/swine/Scotland/410440/1994-like H1ₕᵤN2 (Scot/94) lineage from strain A/swine/Italy/3033-1/2015 (H1N2), and
   a second nucleic acid sequence encoding a second HA antigen of IAV-S of the Eurasian avian-like H1ₐᵥN1 (EA) lineage from strain A/swine/Italy/28762-3/2013 (H1N1).
[2] The nucleic acid construct for use as defined in [1], wherein the first HA antigen encoded by the first nucleic acid sequence comprises an amino acid sequence of SEQ ID NO: 3 or an amino acid sequence having at least 85% sequence identity thereof.
[3] The nucleic acid construct for use as defined in [1] or [2], wherein the second HA antigen encoded by the second nucleic acid sequence comprises an amino acid sequence of SEQ ID NO: 6 or an amino acid sequence having at least 90% sequence identity thereof.
[4] A nucleic acid construct for use in the prevention of a disease caused by a Swine influenza A virus in a subject, the nucleic acid construct comprising first and second nucleic acid sequences:
   the first nucleic acid sequence encoding a first hemagglutinin (HA) antigen of a Swine influenza A virus (IAV-S) of the A/swine/Gent/1/1984-like H3N2 (Gent/84) lineage from strain A/swine/Italy/240849/2015 (H3N2), and
   a second nucleic acid sequence encoding a second HA antigen of IAV-S of the A (H1N1) pdm09 (pdm09) lineage from strain A/swine/England/373/2010 (H1N1).
[5] The nucleic acid construct for use as defined in [4], wherein the first HA antigen encoded by the first nucleic acid sequence comprises an amino acid sequence of SEQ ID NO: 9 or an amino acid sequence having at least 95% sequence identity thereof.
[6] The nucleic acid construct for use as defined in [4] or [5], wherein the second HA antigen encoded by the second nucleic acid sequence comprises an amino acid sequence of SEQ ID NO: 12 or an amino acid sequence having at least 95% sequence identity thereof.
[7] An RNA replicon particle comprising the nucleotide construct as defined in any one of [1] to [3].
[8] An RNA replicon particle comprising the nucleotide construct as defined in any one of [4] to [6].
[9] The RNA replicon particle as defined in [7] or [8], which is an alphavirus RNA replicon particle
[10] The RNA replicon particle as defined in [9], which is a Venezuelan Equine Encephalitis Virus (VEEV) alphavirus RNA replicon particle
[11] An immunogenic composition, comprising the RNA replicon particle as defined in any one of [7] to [10].
[12] The immunogenic composition as defined in comprising the RNA replicon particle as defined in [7] and [8].
[13] A vaccine comprising the immunogenic composition as defined in [12].
[14] The vaccine as defined in [13], which is a nonadjuvanted vaccine.
[15] The vaccine as defined in [13], which comprises an adjuvant selected from the group consisting of a biodegradable oil, an oil-in-water emulsion with 2.5-50% (v/v) mineral oil, and a biodegradable oil mixed with an oil-in-water emulsion with 2.5-50% (v/v) mineral oil.
[16] The vaccine as defined in any one of to for use in the prevention of a disease caused by a Swine influenza A virus in a subject.
[17] A method of immunizing a porcine against a swine influenza A virus, the method comprising administering to the porcine an immunologically effective amount of the vaccine as defined in any one of to [16].
[18] A nucleic acid construct comprising first and second nucleic acid sequences:
   the first nucleic acid sequence encoding a first hemagglutinin (HA) antigen of a Swine influenza A virus (IAV-S) of the A/swine/Gent/1/1984-like H3N2 (Gent/84) lineage from strain A/swine/Italy/240849/2015 (H3N2), and
   a second nucleic acid sequence encoding a second HA antigen of IAV-S of the A (H1N1) pdm09 (pdm09) lineage from strain A/swine/England/373/2010 (H1N1).
[19] A nucleic acid construct comprising first and second nucleic acid sequences:
   a first nucleic acid sequence encoding a first HA antigen of IAV-S of the A/swine/Scotland/410440/1994-like H1ₕᵤN2 (Scot/94) lineage from strain A/swine/Italy/3033-1/2015 (H1N2), and
   a second nucleic acid sequence encoding a second HA antigen of IAV-S of the Eurasian avian-like H1ₐᵥN1 (EA) lineage from strain A/swine/Italy/28762-3/2013 (H1N1).

In a third aspect, the present invention provides the following embodiments:
[1] An immunogenic composition for use in the prevention of a disease caused by a Swine influenza A virus in a subject, the composition comprising first and second RNA replicon particles,
   the first RNA replicon particle comprising a nucleic acid construct, comprising first and second nucleic acid sequences encoding first and second hemagglutinin (HA) antigens of a Swine influenza A virus (IAV-S), wherein
      the first HA antigen is a of the A/swine/Gent/1/1984-like H3N2 (Gent/84) lineage, and
      the second HA antigen is of the A (H1N1) pdm09 (pdm09) lineage,
   the second RNA replicon particle comprising a nucleic acid construct comprising third and fourth nucleic acid sequences encoding third and fourth HA antigens of IAV-S, wherein
      the third HA antigen is of the A/swine/Scotland/410440/1994-like H1ₕᵤN2 (Scot/94) lineage, and
      the fourth HA antigen is of the Eurasian avian-like H1ₐᵥN1 (EA) lineage.
[2] The immunogenic composition for use as defined in [1], wherein the first HA antigen is from strain A/swine/Italy/240849/2015 (H3N2).
[3] The immunogenic composition for use as defined in [1] or [2], wherein the first HA antigen encoded by the first nucleic acid sequence comprises an amino acid sequence of SEQ ID NO: 9 or an amino acid sequence having at least 90% sequence identity thereof.
[4] The immunogenic composition for use as defined in any one of the preceding [1] to [3], wherein the second HA antigen is from strain A/swine/England/373/2010 (H1N1).
[5] The immunogenic composition for use as defined in any one of the preceding [1] to [4], wherein the second HA antigen encoded by the second nucleic acid sequence comprises an amino acid sequence of SEQ ID NO: 12 or an amino acid having at least 95% sequence identity thereof.
[6] The immunogenic composition for use as defined in any one of the preceding [1] to [5], wherein the third HA antigen is from strain A/swine/Italy/3033-1/2015 (H1N2).
[7] The immunogenic composition for use as defined in any one of the preceding [1] to [6], wherein the third HA antigen encoded by the third nucleic acid sequence comprises an amino acid sequence of SEQ ID NO: 3 or an amino acid sequence having at least 85% sequence identity thereof.
[8] The immunogenic composition for use as defined in any one of the preceding [1] to [7], wherein the fourth HA antigen is from strain A/swine/Italy/28762-3/2013 (H1N1).
[9] The immunogenic composition for use as defined in any one of the preceding [1] to [8], wherein the fourth HA antigen encoded by the fourth nucleic acid sequence comprises an amino acid sequence of SEQ ID NO: 6 or an amino acid sequence having at least 90% sequence identity thereof.
[10] The immunogenic composition for use as defined in any one of the preceding [1] to [9], which is adapted for simultaneous administration of the first and second RNA replicon particles.
[11] The immunogenic composition for use as defined in any one of the preceding [1] to [10], further comprising a third RNA replicon particle,
   the third RNA replicon particle comprising a nucleic acid construct, comprising first, second and third nucleic acid sequences encoding first, second and third neuraminidase (NA) antigens of IAV-S, wherein
   the first NA antigen is of the A/swine/Scotland/410440/1994-like H1ₕᵤN2 (Scot/94) lineage,
   the second NA antigen is of the A/swine/Gent/1/1984-like H3N2 (Gent/84) lineage, and
   the third NA antigen is selected from the A (H1N1) pdm09 (pdm09) lineage or the Eurasian avian-like H1ₐᵥN1 (EA) lineage.
[12] The immunogenic composition for use as defined in any one of the preceding [1] to [11], wherein the RNA replicon particle is an alphavirus RNA replicon particle.
[13] The immunogenic composition for use as defined in [12], which is a Venezuelan Equine Encephalitis Virus (VEEV) alphavirus RNA replicon particle.
[14] A vaccine comprising the immunogenic composition as defined in any one of the preceding [1] to [13].
[15] The vaccine as defined in [14], which is a nonadjuvanted vaccine.
[16] The vaccine as defined in [14], which comprises an adjuvant selected from the group consisting of a biodegradable oil, an oil-in-water emulsion with 2.5-50% (v/v) mineral oil, and a biodegradable oil mixed with an oil-in-water emulsion with 2.5-50% (v/v) mineral oil.
[17] The vaccine as defined in any one of to for use in the prevention of a disease caused by a Swine influenza A virus in a subject.
[18] A method of immunizing a porcine against a swine influenza A virus, the method comprising administering to the porcine an immunologically effective amount of the vaccine as defined in any one of to [16].
[19] An immunogenic composition comprising first and second RNA replicon particles,
   the first RNA replicon particle comprising a nucleic acid construct, comprising first and second nucleic acid sequences encoding first and second hemagglutinin (HA) antigens of a Swine influenza A virus (IAV-S), wherein
      the first HA antigen is a of the A/swine/Gent/1/1984-like H3N2 (Gent/84) lineage, and
      the second HA antigen is of the A (H1N1) pdm09 (pdm09) lineage,
   the second RNA replicon particle comprising a nucleic acid construct comprising third and fourth nucleic acid sequences encoding third and fourth HA antigens of IAV-S, wherein
      the third HA antigen is of the A/swine/Scotland/410440/1994-like H1ₕᵤN2 (Scot/94) lineage, and
      the fourth HA antigen is of the Eurasian avian-like H1ₐᵥN1 (EA) lineage.
[20] An immunogenic composition comprising first, second and third RNA replicon particles,
   the first RNA replicon particle comprising a nucleic acid construct, comprising first and second nucleic acid sequences encoding first and second hemagglutinin (HA) antigens of a Swine influenza A virus (IAV-S), wherein
      the first HA antigen is a of the A/swine/Gent/1/1984-like H3N2 (Gent/84) lineage, and
      the second HA antigen is of the A (H1N1) pdm09 (pdm09) lineage,
   the second RNA replicon particle comprising a nucleic acid construct comprising third and fourth nucleic acid sequences encoding third and fourth HA antigens of IAV-S, wherein
      the third HA antigen is of the A/swine/Scotland/410440/1994-like H1ₕᵤN2 (Scot/94) lineage, and
      the fourth HA antigen is of the Eurasian avian-like H1ₐᵥN1 (EA) lineage,
   the third RNA replicon particle comprising a nucleic acid construct, comprising first, second and third nucleic acid sequences encoding first, second and third neuraminidase (NA) antigens of IAV-S, wherein
   the first NA antigen is of the A/swine/Scotland/410440/1994-like H1ₕᵤN2 (Scot/94) lineage,
   the second NA antigen is of the A/swine/Gent/1/1984-like H3N2 (Gent/84) lineage, and
   the third NA antigen is selected from the A (H1N1) pdm09 (pdm09) lineage or the
   Eurasian avian-like H1ₐᵥN1 (EA) lineage.

In a fourth aspect, the present invention provides the following embodiments:
[1] A nucleic acid construct for use in the prevention of a disease caused by a Swine influenza A virus in a subject, the nucleic acid construct comprising first, second and third nucleic acid sequences encoding first, second and third neuraminidase (NA) antigens of a Swine influenza A virus (IAV-S), wherein
   the first NA antigen is of the A/swine/Scotland/410440/1994-like H1ₕᵤN2 (Scot/94) lineage,
   the second NA antigen is of the A/swine/Gent/1/1984-like H3N2 (Gent/84) lineage, and
   the third NA antigen is selected from the A (H1N1) pdm09 (pdm09) lineage or the Eurasian avian-like H1ₐᵥN1 (EA) lineage.
[2] The nucleic acid construct for use as defined in [1], wherein the first NA antigen is from strain A/swine/England/61470/2013 (H1N2).
[3] The nucleic acid construct for use as defined in [1] or [2], wherein the first NA antigen encoded by the first nucleic acid sequence comprises an amino acid sequence of SEQ ID NO: or an amino acid sequence having at least 90% sequence identity thereof.
[4] The nucleic acid construct for use as defined in any one of [1] to [3], wherein the second NA antigen is from strain A/swine/Italy/248147-8/2015 (H3N2).
[5] The nucleic acid construct for use as defined in any one of [1] to [4], wherein the second NA antigen encoded by the second nucleic acid sequence comprises an amino acid sequence of SEQ ID NO: 18 or an amino acid sequence having at least 90% sequence identity thereof.
[6] The nucleic acid construct for use as defined in any one of [1] to [5], wherein the third NA antigen is from strain A/swine/England/373/2010 (H1N1) or A/swine/Italy/179057/2015 (H1N1).
[7] The nucleic acid construct for use as defined in any one of [1] to [6], wherein the third NA antigen is from strain A/swine/Italy/28762-3/2013 (H1N1).
[8] The nucleic acid construct for use as defined in any one of [1] to [7], wherein the third NA antigen encoded by the third nucleic acid sequence comprises an amino acid sequence of SEQ ID NO: 24 or an amino acid sequence having at least 90% sequence identity thereof.
[9] An RNA replicon particle comprising the nucleic acid construct as defined in any one of [1] to [8].
[10] The RNA replicon particle as defined in [9], which is an alphavirus RNA replicon particle.
[11] The RNA replicon particle as defined in [9] or [10], which is a Venezuelan Equine Encephalitis Virus (VEEV) alphavirus RNA replicon particle.
[12] An immunogenic composition comprising the RNA replicon particle as defined in any one of [9] to [11].
[13] An immunogenic composition, comprising first, second and third RNA replicon particles,
   the first RNA replicon particle comprising a nucleic acid construct, comprising first and second nucleic acid sequences encoding first and second hemagglutinin (HA) antigens of a Swine influenza A virus (IAV-S), wherein
      the first HA antigen is of the A/swine/Gent/1/1984-like H3N2 (Gent/84) lineage, and
      the second HA antigen is of the A (H1N1) pdm09 (pdm09) lineage,
   the second RNA replicon particle comprising a nucleic acid construct comprising third and fourth nucleic acid sequences encoding third and fourth HA antigens of IAV-S, wherein
      the third HA antigen is of the A/swine/Scotland/410440/1994-like H1ₕᵤN2 (Scot/94) lineage, and
      the fourth HA antigen is of the Eurasian avian-like H1ₐᵥN1 (EA) lineage, and
   the third RNA replicon particle is the RNA replicon particle as defined in any one of [9] to [11].
[14] A vaccine comprising the immunogenic composition as defined in or [13].
[15] The vaccine as defined in [14], which is a nonadjuvanted vaccine.
[16] The vaccine as defined in [14], which comprises an adjuvant selected from the group consisting of a biodegradable oil, an oil-in-water emulsion with 2.5-50% (v/v) mineral oil, and a biodegradable oil mixed with an oil-in-water emulsion with 2.5-50% (v/v) mineral oil.
[17] The vaccine as defined in any one of to for use in the prevention of a disease caused by a Swine influenza A virus in a subject.
[18] A method of immunizing a porcine against a swine influenza A virus, the method comprising administering to the porcine an immunologically effective amount of the vaccine as defined in any one of to [16].
[19] A nucleic acid construct comprising first, second and third nucleic acid sequences encoding first, second and third neuraminidase (NA) antigens of a Swine influenza A virus (IAV-S), wherein
   the first NA antigen is of the A/swine/Scotland/410440/1994-like H1ₕᵤN2 (Scot/94) lineage,
   the second NA antigen is of the A/swine/Gent/1/1984-like H3N2 (Gent/84) lineage, and
   the third NA antigen is selected from the A (H1N1) pdm09 (pdm09) lineage or the Eurasian avian-like H1ₐᵥN1 (EA) lineage.

The following examples serve to provide further appreciation of the invention but are not meant in any way to restrict the effective scope of the invention.

### EXAMPLES

### Materials and Methods

### Preparation of Alphavirus RNA RP Vaccine

### Generation of Single HA or NA Gene Replicon Particles (RPs).

The VEE replicon vectors designed to express haemagglutinin (HA) or neuraminidase (NA) genes were constructed as previously described [see, U.S. Pat. No. 9,441,247 B2; the contents of which are hereby incorporated herein by reference], with the following modifications. The TC-83-derived replicon vector “pVEK” [disclosed and described in U.S. Pat. No. 9,441,247 B2] was digested with restriction enzymes Ascl and Pad. A DNA plasmid containing the codon-optimized open reading frame sequence of HA or NA genes (Table 1a and 1b) with 5′-flanking sequence (5′-GGCGCGCCGCACC-3′) (SEQ ID NO: 25) and 3′-flanking sequence (5′-TTAATTAA-3′), was similarly digested with restriction enzymes Ascl and Pad. The synthetic gene cassette was then ligated into the digested pVEK vector, and the resulting clones were re-named “pVHV-respective RP code. The “pVHV” vector nomenclature was chosen to refer to pVEK-derived replicon vectors containing transgene cassettes cloned via the Ascl and Pad sites in the multiple cloning site of pVEK.

Production of TC-83 RNA replicon particles (RP) was conducted according to methods previously described [U.S. Pat. No. 9,441,247 B2 and U.S. Pat. No. 8,460,913 B2; the contents of which are hereby incorporated herein by reference]. Briefly, pVHV replicon vector DNA and helper DNA plasmids were linearized with Not1 restriction enzyme prior to in vitro transcription using MegaScript T7 RNA polymerase and cap analog (Promega, Madison, WI). Importantly, the helper RNAs used in the production lack the VEE subgenomic promoter sequence, as previously described [Kamrud et al., J Gen Virol. 91 (Pt 7): 1723-1727 (2010)]. Purified RNA for the replicon and helper components were combined and mixed with a suspension of Vero cells, electroporated in 4 mm cuvettes, and returned to OptiPro SFM cell culture media (Thermo Fisher, Waltham, MA). Following overnight incubation, alphavirus RNA replicon particles were purified, formulated in phosphate buffered saline with 5% sucrose (w/v) and 1% swine serum, passed through a 0.22 micron membrane filter, and dispensed into aliquots for storage. Titer of functional RP was determined by immunofluorescence assay on infected Vero cell monolayers. Batches of RP were identified according to the gene encoded by the packaged replicon (Tables 1a&b).

### Generation of Multi HA or NA Genes Replicon Particles (RPs).

The VEE replicon vectors used to express HA or NA genes were constructed as previously described [see, U.S. Pat. No. 9,441,247 B2; the contents of which are hereby incorporated herein by reference], with the following modifications. The TC-83-derived replicon vector “pVEK” [disclosed and described in U.S. Pat. No. 9,441,247 B2] was digested with restriction enzymes Ascl and PacI. For dual-gene HA and NA constructs, the selected open reading frame sequences were codon-optimized and synthesized with flanking Ascl and PacI sites. Furthermore, the interstitial sequence between the two synthetic HA or NA open-reading frames consisted of 47 nucleotides of non-coding heterologous sequence, and a second copy of the native TC-83 subgenomic (sg) RNA promoter and 5′ untranslated sgRNA region sequence. These dual-gene constructs were termed “pVDG” to differentiate them from the parental vector with a single sgRNA promoter sequence. In case of triple-gene NA constructs, the pVDG-based constructs containing two NA genes was further modified, as follows. A third selected NA open reading frame was codon optimized and synthesized with flanking PacI and SphI sites for directional cloning into the pVDG vector downstream of the two existing NA genes. The new synthetic construct also contained 50 nucleotides of heterologous non-coding sequence, and a third copy of the native TC-83 sgRNA promoter and 5′ untranslated sgRNA region sequence to the 5′ of the third NA gene sequence. The 3′ region from the third NA gene sequence consisted of the 3′ untranslated region of TC-83, until the corresponding SphI site of the parental pVDG vector. The triple-gene vectors were termed “pVTG” to differentiate them from related vectors pVEK, pVHV, and pVDG.

The selected sequences of HA (Tablela: EUHA1-3, EUHA1-2, EUHA1-5, EUHA1-15, EUHA1-17, EUHA1-8, EUHA1-11 and HA3-4) or NA (Table 1b: EUNA1-2, EUN1-4, EUN2-6 and EUN2-7) genes from EXAMPLES 1 & 3 were used to synthesize the multi-HA or NA genes in the plasmid vector pVDG or pVTG, as described above.

Production of TC-83 RNA replicon particles (RP) was conducted according to methods previously described [U.S. Pat. No. 9,441,247 B2 and U.S. Pat. No. 8,460,913 B2; the contents of which are hereby incorporated herein by reference]. Briefly, pVDG or pVTG replicon vector DNA and helper DNA plasmids were linearized with Not restriction enzyme prior to in vitro transcription using MegaScript T7 RNA polymerase and cap analog. Importantly, the helper RNAs used in the production lack the VEE subgenomic promoter sequence, as previously described [Kamrud et al., J Gen Virol. 91 (Pt 7): 1723-1727 (2010)]. Purified RNA for the replicon and helper components were combined and mixed with a suspension of Vero cells, electroporated in 4 mm cuvettes, and returned to serum-free culture media. Following overnight incubation, alphavirus RNA replicon particles were purified from the cells and media by passing the suspension through a depth filter, washing with phosphate buffered saline containing 5% sucrose (w/v), and finally eluting the retained RP with 200 mM Na₂SO₄+5% sucrose (w/v) buffer. Alternatively, the cells and media were centrifuged in the presence of prepared Cellufine Sulfate® resin, washed with phosphate buffered saline containing 5% sucrose (w/v), and eluted with 200 mM Na2SO4+5% sucrose (w/v) buffer.

Eluted RP were passed through a 0.22 micron membrane filter, and dispensed into aliquots for storage. Titer of functional RP was determined by immunofluorescence assay on infected Vero cell monolayers.

The following replicon particles were constructed and used in the experiments:

TABLE 1a
RNA particles, HA source strains, lineages, clade and GenBank accession numbers (Accession#)

| RP code | RP donor strain | Lineage | Clade | Accession # |
|---|---|---|---|---|
| EUH1-1 | A/swine/Denmark/10802-1/2012(H1N2) | EurAsianAvian(EA) | 1C.2-like | AKC43997.1 |
| EUH1-2 | A/swine/Denmark/10-1048-2/2011(H1N1) | EurAsianAvian(EA) | 1C.2 | AKC43996.1 |
| EUH1-3 | A/swine/Italy/28762-3/2013(H1N1) | EurAsianAvian(EA) | 1C.2.1 | AKJ81667.1 |
| EUH1-4 | A/swine/France/29-120326/2012(H1N1) | EurAsianAvian(EA) | 1C.2.1 | AKJ82257.1 |
| EUH1-5 | A/swine/France/44-120070/2012(H1N1) | EurAsianAvian(EA) | 1C.2.1 | AIL24876.1 |
| EUH1-13 | A/swine/France/56-140048-2014(H1N1) | EurAsianAvian(EA) | 1C.2.1 | AJW32121.1 |
| EUH1-9 | A/swine/Moeglingen/IDT14859/2012(H1N2) | Pandemic 2009 (pdm09) | 1A.3.3.2 | AGG86840.1 |
| EUH1-11 | A/swine/England/373/2010(H1N1) | Pandemic 2009 (pdm09) | 1A.3.3.2 | AFR76205.1 |
| EUH1-12 | A/swine/Italy/290271/2009(H1N1) | Pandemic 2009 (pdm09) | 1A.3.3.2 | ADA70669.1 |
| EUH1-8 | A/swine/England/9953/2012(H1N2) | Scotland 1994-clade 1 | 1B.1.1 | AKJ81533.1 |
| | | (Scot94-1) | | |
| EUH1-6 | A/swine/Italy/186822/2011(H1N2) | Scotland 1994-clade 2 | 1B.1.2.2 | AGR45140.1 |
| | | (Scot94-2.2) | | |
| EUH1-17 | A/swine/Italy/3033-1/2015(H1N2) | Scotland 1994-clade 2 | 1B.1.2.2 | ALX30160.1 |
| | | (Scot94-2.2) | | |
| EUH1-7 | A/swine/France/22-130212/2013(H1N2) | Scotland 1994-clade 3 | 1B.1.2.3 | AHI43247.1 |
| | | (Scot94-2.3) | | |
| EUH1-15 | A/swine/France/35-140041/2014(H1N2) | Scotland 1994-clade 3 | 1B.1.2.3 | AIL24895.1 |
| | | (Scot94-2.3) | | |
| EUH3-1 | A/swine/Netherlands/Ysselsteyn- | Gent 1984 (Gent/84) | 3.1970.1 | AKJ83041.1 |
| | CVI8864A/2012(H3N2) | | | |
| EUH3-2 | A/swine/Belgium/Glabbeek- | Gent 1984 (Gent/84) | 3.1970.1 | AKJ82900.1 |
| | 284/2012(H3N2) | | | |
| EUH3-3 | A/swine/Spain/33936/2012(H3N2) | Gent 1984 (Gent/84) | 3.1970.1 | AKJ83006.1 |
| EUH3-4 | A/swine/Italy/240849/2015(H3N2) | Gent 1984 (Gent/84) | 3.1970.1 | ALX30415.1 |

TABLE 1b
RNA particles, NA source strains, lineages
and GenBank accession number (Accession#)

| RP code | RP donor strain | Lineage | Accession # |
|---|---|---|---|
| EUN1-2 | A/swine/Italy/28762-3/2013(H1N1) | EA | AKJ81669.1 |
| EUN1-3 | A/swine/Denmark/101048-2/ | EA | AKC43999.1 |
| | 2011(H1N1) | | |
| EUN1-1 | A/swine/England/373/2010(H1N1) | Pdm09 | AFR76207.1 |
| EUN1-4 | A/swine/Italy/179057/2015(H1N1) | Pdm09 | ALX30323.1 |
| EUN2-1 | A/swine/Denmark/10801-2/2012(H1N2) | Scot94 | AKC44047.1 |
| EUN2-3 | A/swine/England/9953/2012(H1N2) | Scot94 | AKJ81535.1 |
| EUN2-5 | A/swine/Italy/246087/2014(H1N2) | Scot94 | ALX29925.1 |
| EUN2-6 | A/swine/England/61470/2013(H1N2) | Scot94 | AKJ82042.1 |
| EUN2-2 | A/swine/Netherlands/Ysselsteyn- | Gent84 | AKJ83043.1 |
| | CVI8864A/2012(H3N2) | | |
| EUN2-7 | A/swine/Italy/248147-8/2015(H3N2) | Gent84 | ALX30429.1 |

Note:
Clade information is not available for NA antigens

If not indicated otherwise in the Examples or Figures, the following strains and lineages have been used for HI assays:

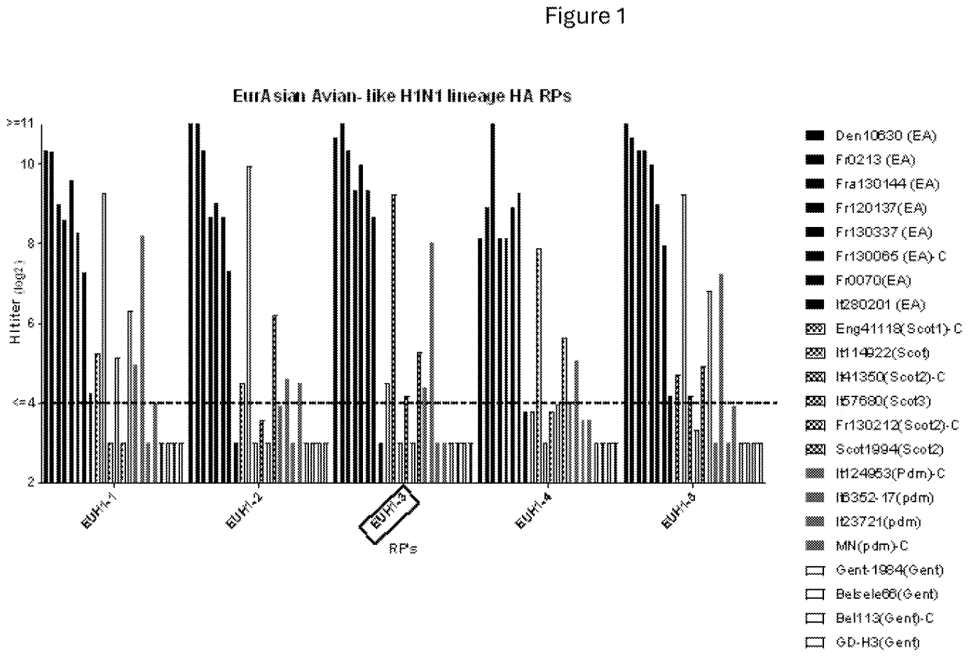

TABLE 1c
SIV strains used as HA antigen source for HI assays in FIGS. 1, 2, 3 and 4

| # | SIV strain | Abbrevation | Lineage | Accession # |
|---|---|---|---|---|
| 1 | A/swine/Denmark/10630-1/2009 (H1N2) | Den10630 (EA) | EA | AKC43951.1 |
| 2 | A/swine/France/Morbihan-0213/2011 (H1N2) | Fr0213 (EA) | EA | AKJ80535.1 |
| 3 | A/swine/France/70-130144/2013 (H1N1) | Fra130144 (EA) | EA | AIL24884 |
| 4 | A/swine/France/41-120137/2012 (H1N1) | Fr120137(EA) | EA | AIL24877.1 |
| 5 | A/swine/France/50-130337/2013 (H1N1) | Fr130337 (EA) | EA | AIL24889.1 |
| 6 | A/swine/France/53-130065/2013 (H1N1) | Fr130065 (EA)-C | EA | AGL07471.1 |
| 7 | A/swine/Morbihan/0070/2005 (H1N1) | Fr0070 (EA) | EA | Not available |
| 8 | A/swine/Italy/280201/2013 (H1N1) | It280201 (EA) | EA | ALX29477.1 |
| 9 | A/swine/Deventer/1/2012 (H1N1) | GD-H1 (EA) | EA | Not available |
| 10 | A/swine/England/041118/2013 (H1N2) | Eng41118 (Scot1)-C | Scot94-1 | AKJ81500.1 |
| 11 | A/swine/Italy/114922/2014 (H1N2) | It114922 (Scot2) | Scot94-2 | ALX29748.1 |
| 12 | A/swine/Italy/41350/2011 (H1N2) | It41350(Scot2)-C | Scot94-2 | AKJ80629.1 |
| 13 | A/swine/Italy/57680/2011 (H1N2) | It57680 (Scot3) | Scot94-3 | AKJ80640.1 |
| 14 | A/swine/France/22-130212/2013 (H1N2) | Fr130212 (Scot2)-C | Scot94-2 | AHI43247.1 |
| 15 | A/swine/Scotland/410440/94 (H1N2) | Scot1994 (Scot2) | Scot94-2 | Not available |
| 16 | A/swine/Gent/7625/1999 (H1N2) | GD-H1 (Scot3) | Scot94-3 | AZQ96426.1 |
| 17 | A/swine/Italy/124953/2014 (H1N2) | It24953 (pdm)-C | Pdm09 | ALX29759.1 |
| 18 | A/swine/Italy/6352-17/13 (H1N1) | It6352-17 (pdm) | Pdm09 | AIC64646.1 |
| 19 | A/swine/Italy/23721/2015 (H1N1) | It23721 (pdm) | Pdm09 | ALX30205.1 |
| 20 | A/swine/MN/A01483170/2014 (H1N1) | MN (pdm)-C | Pdm09 | AIX94905.1 |
| 21 | A/swine/Belgium/Gent-1/1984 (H3N2) | Gent-1984 (Gent) | Gent84 | AKJ82853.1 |
| 22 | A/swine/Belgium/Belsele-66/2013 (H3N2) | Belsele66 (Gent) | Gent84 | AKJ82983.1 |
| 23 | A/swine/Belgium/113/2013 (H3N2) | Bel113 (Gent)-C | Gent84 | AKJ82888.1 |
| 24 | A/swine/Flanders/1/1998 (H3N2) | GD-H3 (Gent) | Gent84 | ACN98096.1 |

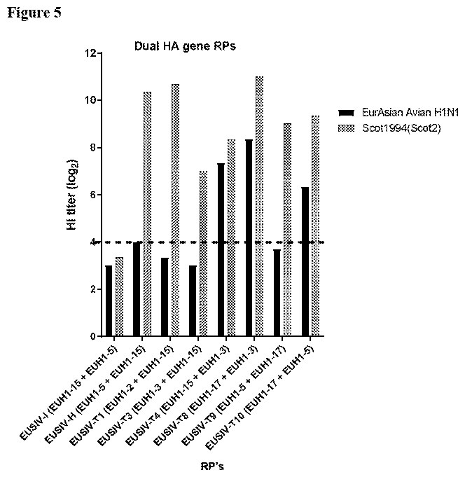

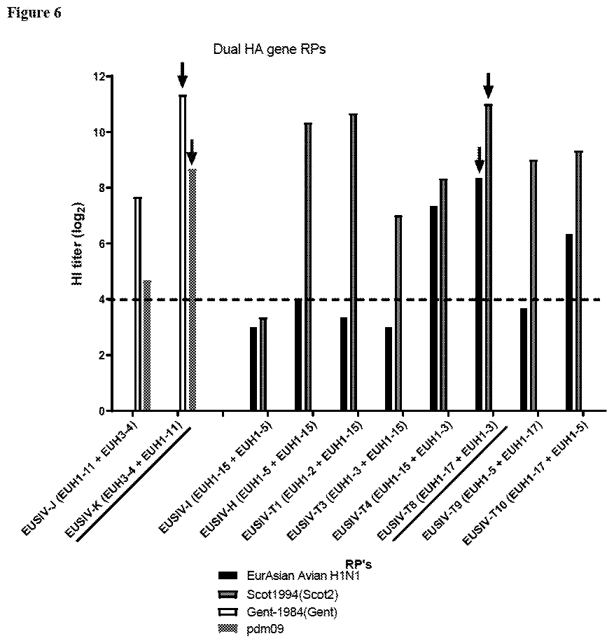

TABLE 1d
SIV strains used as HA antigen source
for HI assays in FIGS. 5 and 6

| # | Strain name | HA Lineage |
|---|---|---|
| 1 | A/swine/France/53-130065/2013 (H1N1) | EA |
| 2 | A/swine/Belgium/113/2013 (H3N2) | Gent/84 |
| 3 | A/swine/MN/A01483170/2014 (H1N1) | pdm09 |
| 4 | A/swine/England/041118/2013 (H1N2) | Scot94-1 |
| 5 | A/swine/France/22-130212/2013 (H1N2) | Scotland 1994-2 |
| | | (Scot94-2) |

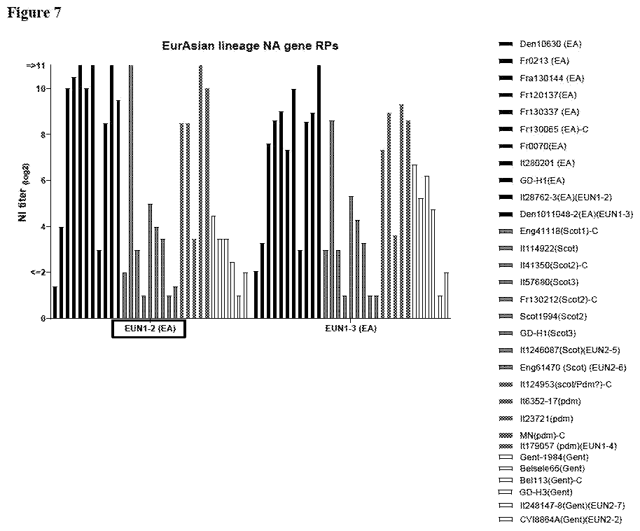

TABLE 1e
SIV strain or expressed NA antigen source for NI assays in FIGS. 7, 8, 9 and 10

| # | SIV strain or expressed NA protein | Abbrevation | Lineage | Accession # |
|---|---|---|---|---|
| 1 | A/swine/Denmark/10630-1/2009 (H1N2) | Den10630 (EA) | EA | AKC44059.1 |
| 2 | A/swine/France/Morbihan-0213/2011 (H1N2) | Fr0213 (EA) | EA | AKJ80537.1 |
| 3 | A/swine/France/70-130144/2013 (H1N1) | Fra130144 (EA) | EA | Not available |
| 4 | A/swine/France/41-120137/2012 (H1N1) | Fr120137(EA) | EA | Not available |
| 5 | A/swine/France/50-130337/2013 (H1N1) | Fr130337 (EA) | EA | Not available |
| 6 | A/swine/France/53-130065/2013 (H1N1) | Fr130065 (EA)-C | EA | AGL07474.1 |
| 7 | A/swine/Morbihan/0070/2005 (H1N1) | Fr0070 (EA) | EA | Not available |
| 8 | A/swine/Italy/280201/2013 (H1N1) | It280201 (EA) | EA | ALX29479.1 |
| 9 | A/swine/Deventer/1/2012 (H1N1) | GD-H1 (EA) | EA | Not available |
| 10 | A/swine/Italy/28762-3/2013 (H1N1) | It28762-3 (EA) EUN1-2* | EA | AKJ81669.1 |
| 11 | A/swine/Denmark/101048-2/2011 (H1N1) | Den1011048 (EA) EUN1-3* | EA | AKC43999.1 |
| 12 | A/swine/England/041118/2013 (H1N2) | Eng41118 (Scot)-C | Scot94 | AKJ81502.1 |
| 13 | A/swine/Italy/114922/2014 (H1N2) | It114922 (Scot) | Scot94 | ALX29750.1 |
| 14 | A/swine/Italy/41350/2011 (H1N2) | It41350(Scot)-C | Scot94 | AFU09071.1 |
| 15 | A/swine/Italy/57680/2011 (H1N2) | It57680 (Scot) | Scot94 | AKJ80642.1 |
| 16 | A/swine/France/22-130212/2013 (H1N2) | Fr130212 (Scot)-C | Scot94 | AHI43225.1 |
| 17 | A/swine/Scotland/410440/94 (H1N2) | Scot1994 (Scot) | Scot94 | CAC86322.1 |
| 18 | A/swine/Gent/7625/1999 (H1N2) | GD-H1 (Scot) | Scot94 | AZQ96428.1 |
| 19 | A/swine/Italy/246087/2014 (H1N2) | It246087 (Scot) EUN2-5* | Scot94 | ALX29925.1 |
| 20 | A/swine/England/61470/2013 (H1N2) | Eng61470 (Scot) EUN2-6* | Scot94 | AKJ82042.1 |
| 21 | A/swine/Italy/124953/2014 (H1N2) | It124953 (Scot)-C | Scot94 | ALX29761.1 |
| 22 | A/swine/Italy/6352-17/13 (H1N1) | It6352-17 (pdm) | pdm09 | AIC64648.1 |
| 23 | A/swine/Italy/23721/2015 (H1N1) | It23721 (pdm) | pdm09 | ALX30207.1 |
| 24 | A/swine/MN/A01483170/2014 (H1N1) | MN (pdm)-C | pdm09 | AIX94908.1 |
| 25 | A/swine/Italy/179057/2015 (H1N1) | It179057 (pdm) EUN1-4* | pdm09 | ALX30323.1 |
| 26 | A/swine/Belgium/Gent-1/1984 (H3N2) | Gent-1984 (Gent) | Gent84 | Not available |
| 27 | A/swine/Belgium/Belsele-66/2013 (H3N2) | Belsele66 (Gent) | Gent84 | AKJ82985.1 |
| 28 | A/swine/Belgium/113/2013 (H3N2) | Bel113 (Gent)-C | Gent84 | AKJ82890.1 |
| 29 | A/swine/Flanders/1/1998 (H3N2) | GD-H3 (Gent) | Gent84 | Not available |
| 30 | A/swine/Italy/248147-8/2015 (H3N2) | It248147 (Gent) EUN2-7* | Gent84 | ALX30429.1 |
| 31 | A/swine/Netherlands/Ysselsteyn- | CVI8864A (Gent) EUN2-2* | Gent84 | AKJ83043.1 |
| | CVI8864A/2012 (H3N2) | | | |

*Lysates of vero cells expressing respective NA antigen was used as source of NA antigen

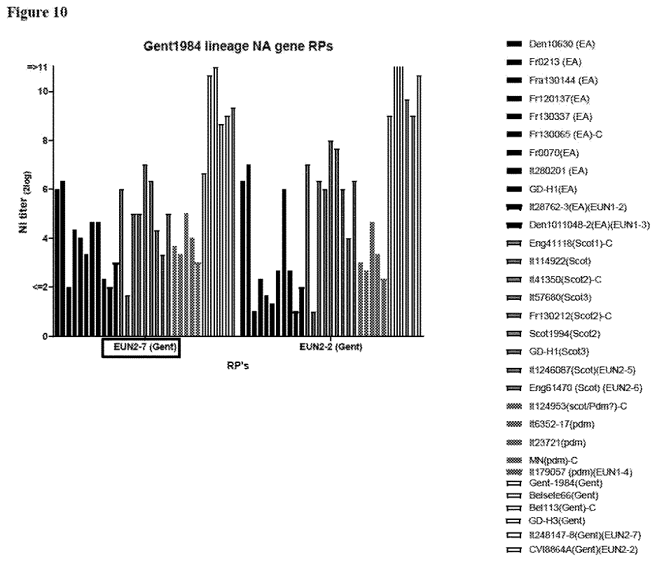

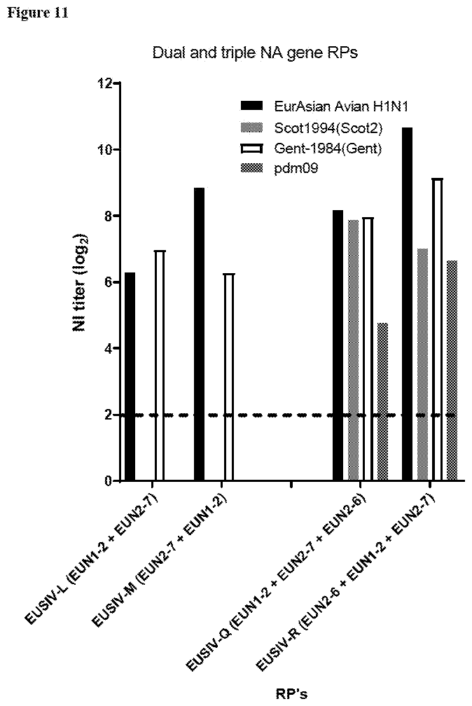

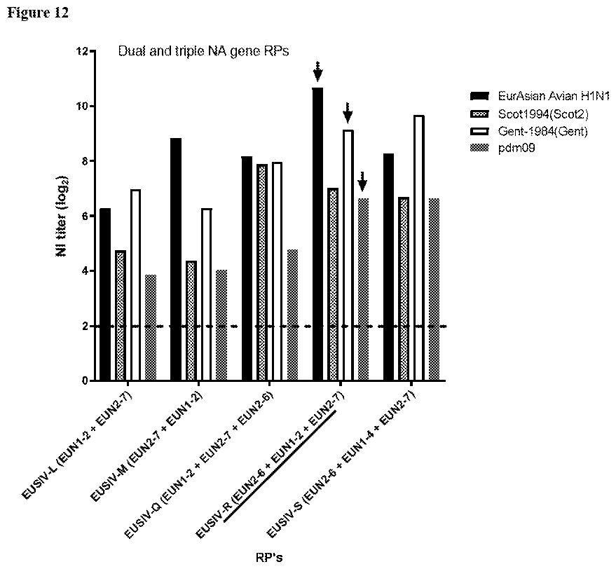

TABLE 1f
NA antigen source for NI assays in FIGS. 11 and 12

| # | NA donor strain | Abbrevation | Lineage | Accession # |
|---|---|---|---|---|
| 1 | A/swine/Italy/28762-3/2013 (H1N1) | It28762-3 (EA) EUN1-2* | EA | AKJ81669.1 |
| | A/swine/Italy/179057/2015 (H1N1) | It179057 (pdm) EUN1-4* | pdm09 | ALX30323.1 |
| 4 | A/swine/England/61470/2013 (H1N2) | Eng61470 (Scot) EUN2-6* | Scot94 | AKJ82042.1 |
| 6 | A/swine/Italy/248147-8/2015 (H3N2) | It248147 (Gent) EUN2-7* | Gent84 | ALX30429.1 |

*Lysates of vero cells expressing respective NA antigen was used as source of NA antigen

### General Study Design

Approximately, five weeks old healthy pigs (3 pigs per vaccine) that were seronegative or had low antibodies against SIV were vaccinated intramuscularly with 5-10×10⁶ RNA particle vaccines encoding either single or multiple HA or NA genes with Xsolve50 adjuvant per pig per time. The respective vaccination was repeated at approximately 8 weeks of age and the blood samples were collected approximately 9 weeks of age and were used for either Hemagglutination inhibition (HI) assay or Neuraminidase inhibition (NI) assays to quantify the levels of antigen specific antibody levels.

Hemagglutination inhibition (HI) assay:

All serum samples were heat inactivated at 56° C. for 30 min, subsequently treated with 0.25% periodate, followed by 0.75% glycerol and adsorbed with 2.6% chicken red blood cells to remove non specific agglutinins. For HI antibody titration, serial dilutions of pre-treated serum were incubated for 1 hour with 8 hemagglutination units of SIV strains listed in table 1c or 1d as HA antigens. Subsequently, the mixture was incubated with 0.2% chicken red blood cells for 1 hour at room temperature and plates were read for inhibition of agglutination. The reciprocal of the highest serum dilution that completely inhibited erythrocyte agglutination was assigned as the HI titer and expressed in log base 2 values.

Serum neuraminidase (NA) inhibition (NI) assay:

SIV strains of lysates of Vero cell expressing NA antigens electroporating the with replicon RNA encoding genes for respective NA (Tables 1e and 1f) were used as source of NA antigens. Enzymatic activity of those NA was quantified by sialic acid cleavage from fetuin on 96-well plates during an overnight incubation at 37° C. Peanut agglutinin-horseradish peroxidase conjugate (PNA-HRP) was then added for 2 h at room temperature, binding to fetuin molecules stripped of sialic acid. Signal was obtained with 3,3′,5,5′-Tetramethylbenzidine (TMB) substrate and read at 450 nm. Test antigens were titrated to determine the dilution that is able to yield 70% of the maximum signal. Equal volumes of NA antigen were added to serial dilutions of serum in fetuin-coated wells during the overnight 37° C. incubation. Optical density (OD) values were normalized to the values from positive control wells containing no serum. Neuraminidase inhibition titers were defined as the reciprocal of the interpolated serum dilution having an extinction value equal to 50% inhibition in comparison with the control and were expressed in log base 2 values.

The correlation of neuraminidase and hemagglutinin antibody titers with vaccine-induced protection against SIV-A is described in: [Hobson D. et al., J Hyg (Lond) 70, 767-777 (1972); Ohmit S E, et al., J. Infect. Dis 204, 1879-1885 (2011); Walz L, et al., J Virol. 2018; 92 (17): e01006-18. (2018). Thus, the serology results in the Hi and Ni inhibition assays described in the following Examples are indicative of the prevention of disease caused by SIV-A.

### EXAMPLE 1: Hemagglutination Inhibition (HI) Antibody Titers Induced by RP Encoding Single HA Antigens

In order to determine protection and cross-protection of alphavirus RNA RP encoding single HA antigens of each of the strains EurAsianAvian (EA), Gent/84, Scot/94 and pdm09 the following study was carried out:

Five weeks old pigs (3 per group) were vaccinated with respective RNA particle with XSolve50 adjuvant in a prime-boost regimen with approximately 3 weeks interval. Sera were collected one to two weeks post booster vaccination to determine influenza antigen specific hemagglutination inhibition antibody titers, a correlate of protection against influenza. The HI assay measures the highest dilution of serum that prevents influenza virus-induced hemagglutination of erythrocytes. The reciprocal of this dilution was defined as the HI titer in Log 2 base. The reported values are average of 3 animals. The detection limit for this assay is 4 (dotted line in the figures) and hence the titer below 4 are reported as 3 in the figures.

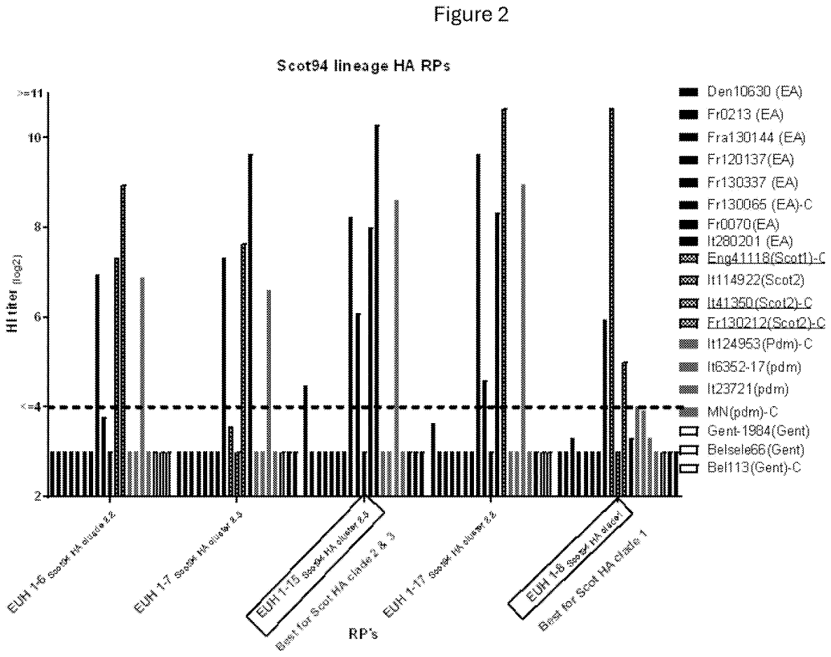

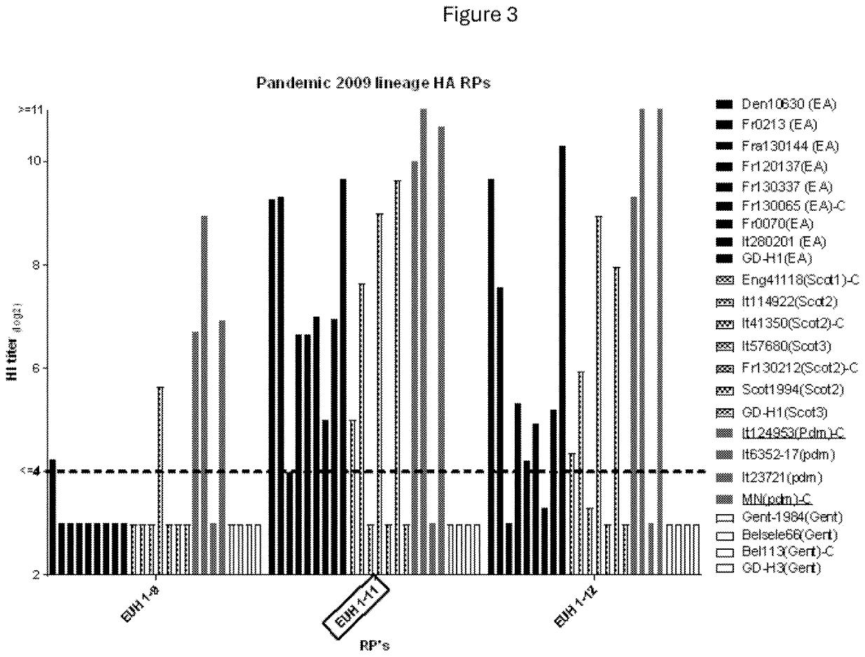

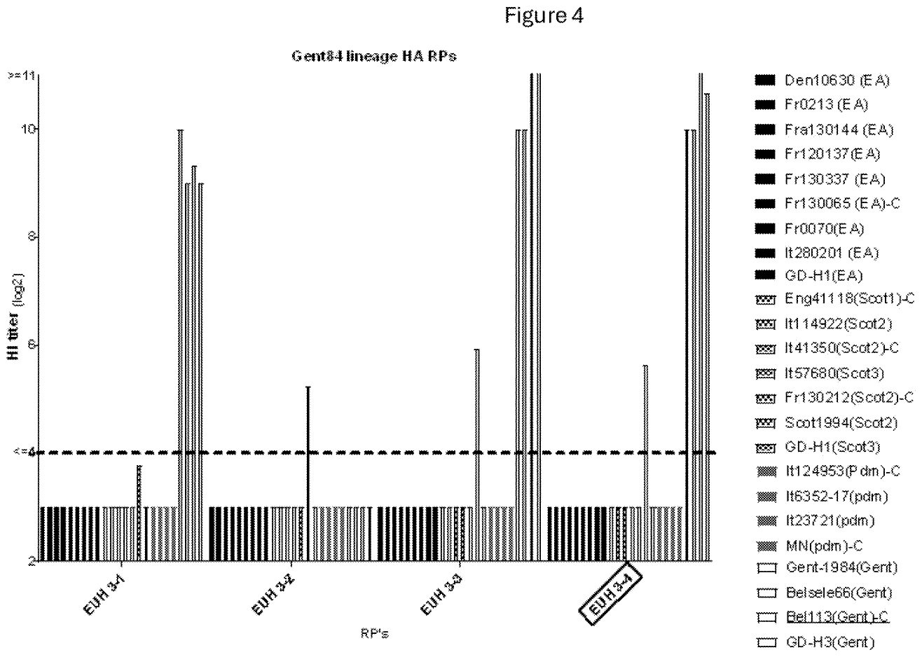

The results of HI experiments are shown in FIGS. 1-4. The following conclusions could be drawn:
FIG. 1: RPs of strain EUHA1-3 of the EA lineage showed highest antigen specific HI antibody titers against nearly all tested EA antigens of IAS followed by EUH1-5 and EUH1-2. In addition, cross reactive titers against some Scot/94 and pdm09 HA antigens could be observed. None of the tested strains showed any cross-reactive titers against Gent/84 IAS antigens (all HI titers below 4).
FIG. 2: RPs of strain EUHA1-15 showed highest antigen specific HI antibody titers against nearly all tested Scot/94 antigens followed by EUH1-17, and thus performed best for Scot/94 antigens of clade 2 and 3. RPs of strain EUHA1-8 showed highest antigen specific HI antibody titers against tested Scot/94 antigens of clade 1. In addition, cross reactive titers against some EA and pdm09 HA IAS antigens could be observed. None of the tested strains showed any cross-reactive titers against Gent/84 IAS antigens (all HI titers below 4).
FIG. 3: RPs of strain EUHA1-11 of the Pdm09 lineage showed highest antigen specific HI antibody titers against nearly all pdm09 antigens. In addition, cross reactive titers against most EA and Scot/94HA antigens could be observed. None of the tested strains showed any cross-reactive titers against Gent/84 IAS antigens (all HI titers below 4).
FIG. 4: RPs of strain EUHA3-4 of the Gent/84 lineage showed highest antigen specific HI antibody titers against all tested Gent/84 antigens. No significant cross-reactive titers against HA antigens of EA, Scot/94 and pdm09 antigens could be observed.

### EXAMPLE 2: Hemagglutination Inhibition (HI) Antibody Titers Induced by RP Encoding Dual-HA Antigens

In order to determine serological efficacy of alphavirus RNA RP encoding dual-e HA antigens combining
1) HA antigens of pdm09 and Gent/84 lineage or
2) HA antigens of EA and Scot/94 antigens, a study with a design as described in Example 1 was carried out.

The results of HI assay are shown in FIG. 5. The following conclusions could be drawn:

It could be observed that not all combinations tested induce a strong serological response. In addition, it could surprisingly be observed that the order of the genes in the viral genome of the replicon particle is critical in inducing a serological response.
Combination of HA antigens of lineages Pdm09 and Gent/84: Only the combination with Gent/84 placed first and pdm09 placed second in the viral genome of the replicon particle induced a strong serological response. Instead, in the order of Pdm09 placed first and Gent/84 placed second in the viral genome of the replicon particle only a much lower serological response against Gent/84 HA antigens and a very weak serological response against Pdm09 HA antigens could be observed.
Combination of HA antigens of lineages EA and Scot/94: Not all combinations tested induced a strong serological response. The combination of strains EUHA1-17 of Scot/94 and EUHA1-3 of EA showed the best serological response (highest HI titers against IAS antigens of both lineages).
In addition, only the combination with Scot/94 placed first and EA placed second in the replicon RNA of the replicon particle induced a strong serological response. Instead, in the order of EA placed first and Scot/94 placed second in the replicon RNA of the replicon particle no significant serological response against EA HA antigens could be observed.
Among various combinations tested, the combination of EUHA3-4+EUHA1-11 and EUHA1-17+EUHA1-3 strains induce the best immunity measured as HI titer. Thus, these combinations are beneficially used in a formulation combining the two replicon particles, i.e. for combining a first RNA replicon particle encoding, in this order, EUHA3-4+EUHA1-11 strains with a second RNA replicon particle encoding, in this order, EUHA1-17+EUHA1-3 strains.

In consequence, it could surprisingly be demonstrated that the position of the HA gene within the RNA replicon particle and/or the specific combination of HA antigens determines the level of induced immunity measured as HI titer.

### EXAMPLE 3: Neuraminidase Inhibition (NI) Antibody Titers Induced by RP Encoding Single NA Antigens

In order to determine serological efficacy of alphavirus RNA RP encoding single NA antigens of each of the strains EurAsianAvian (EA), Gent/84, Scot/94 and pdm09 the following study was carried out:

Five weeks old pigs (3 per group) were vaccinated with respective RNA replicon particle with XSolve50 adjuvant in a prime-boost regimen with approximately 3 weeks interval. Sera were collected one to two weeks post booster vaccination to determine influenza antigen specific neuraminidase inhibition (NI) antibody titers. NI titers were measured using the lectin (peanut agglutinin)-based assay as described above, the reciprocal of the highest dilution of serum that inhibits NA activity at least 50% compared to control wells was defined as the NI titer. The detection limit for this assay was 2 (dotted line in the figure).

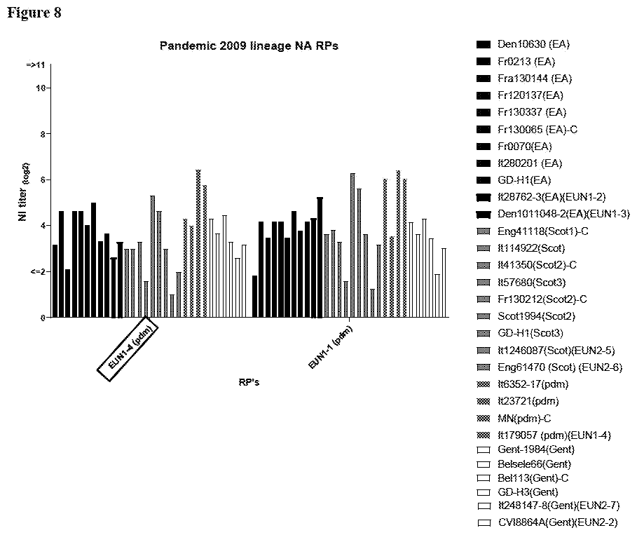

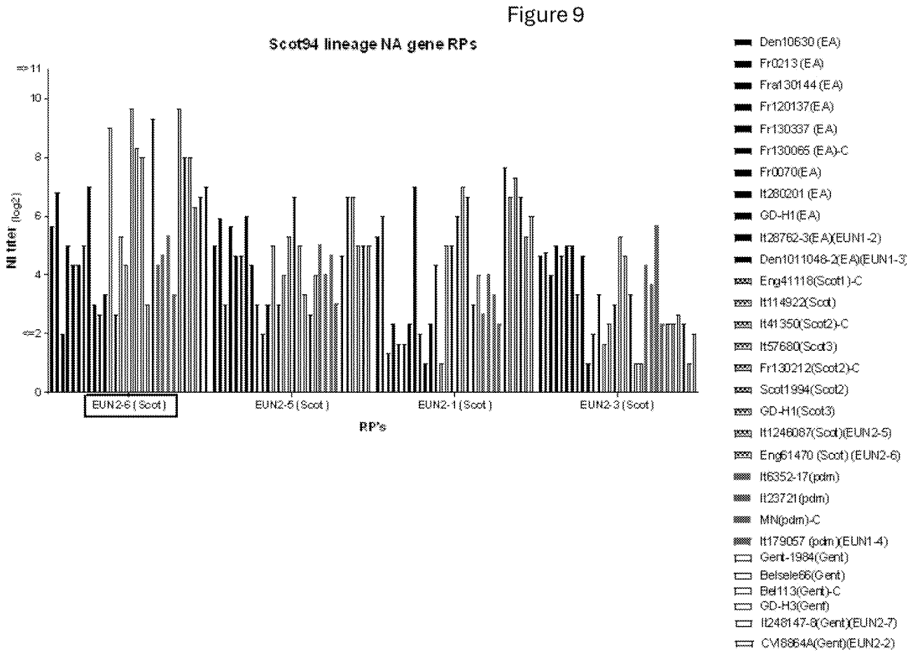

The results of NI experiments are shown in FIGS. 7-10. The following conclusions could be drawn:
FIG. 7: RPs of strain EUNA1-2 of the EA lineage showed highest antigen specific NI antibody titers against nearly all tested EA antigens of IAS. In addition, cross reaction against some Scot/94, pdm09 and Gent/84 NA antigens could be observed.
FIG. 8: RPs of strain EUNA1-4 showed highest antigen specific NI antibody titers against most tested pdm09 antigens, although level of NI titers observed was lower compared to NI titers achieved with RPs of the EA lineage. In addition, cross reactive titer against EA, Scot/94 and Gent/84 NA IAS antigens could be observed. Differences in the titers measured between the tested strains were low.
FIG. 9: RPs of strain EUNA2-6 of the Scot/94 lineage showed highest antigen specific NI antibody titers against all tested Scot/94 antigens. In addition, a high level of cross reactivity against EA, pdm09 and Gent/84 NA antigens could be observed for strain EUNA2-6.
FIG. 10: RPs of strain EUNA2-7 of the Gent/84 lineage showed high antigen specific NI antibody titers against all tested Gent/84 antigens and also showed significant cross-protection against NA antigens of EA, Scot/94 and pdm09 antigens.

### EXAMPLE 4: NI Antibody Titers Induced by RP Encoding Dual- or Triple NA Antigens

In order to determine serological efficacy of alphavirus RNA RP encoding dual- or triple NA antigens, RP encoding NA antigens from below listed lineages were designed and produced
1) NA antigens of EA and Gent/84 lineage or
2) NA antigens of EA, Gent/84 and Scot/94 antigens,
   a study with a design as described in Example 3 was carried out.

The results of NI experiments are shown in FIG. 11. The following conclusions could be drawn:

It could be shown that all combinations tested induce a serological response irrespective of the order of the gene. Thus, it could surprisingly be observed that contrary to the observations with the HA antigens (see Example 2 above), the order of the NA genes in the viral genome of the replicon particle is not critical for inducing a serological response.

### EXAMPLE 5: NI Antibody Titers Induced by RP Encoding Dual- and Triple-NA Antigens

The results shown in FIGS. 7 to 10 reveal that a combination of a strain of the EA lineage, the Gent/84 lineage with strain of the Scot/94 lineage should provide the best protection against IAS having the best protection and cross protection against all four lineages. The best candidate to test such cross-protection is thus a combination of strain EUNA2-6 of the Scot/94 lineage with EUNA2-7 of the Gent/84 lineage, which may then further be combined with strains of either the EA lineage, such as strain EUNA1-2 or the pdm09 lineage, such as strain EUNA1-4. In consequence, these combinations of strains were tested for their serological response.

Thus, in order to determine protection of alphavirus RNA RP encoding dual- and triple-NA antigens combining
1) NA antigens of EA and Gent/84 lineage
2) NA antigens of Scot/94, Gent/84 and EA antigens or
3) NA antigens of Scot/94, Gent/84 and pdm09 antigens,
   a study with a design as described in Example 3 was carried out.

The results are shown in FIG. 12:
Contrary to the results observed for the HA antigens, the combination of NA antigens from only three lineages is sufficient to induce a serological response against all four IAS lineages.
Weak serological response against all four IAS lineages could already be achieved with the combination of NA antigens from only two lineages, irrespective of the order of gene in the RNA replicon particle.
Highest serological response could be achieved with a combination of Scot/94 and Gent/84 NA antigens further combined with either NA antigens of the pdm09 or the EA lineage.

TABLE 2
List of SEQ ID NOs

| SEQ ID NO # | Accession No., Donor strain; RP code | Sequence |
|---|---|---|
| 1 | >KU323133.1 Influenza A virus | Nucleotide, native sequence |
| | (A/swine/Italy/3033-1/2015(H1N2)) segment 4 | |
| | hemagglutinin (HA) gene, complete cds | |
| 2 | >EUH1-17 Swine optimized sequence for EUSIV- | Nucleotide, Codon optimized |
| | T8-RP production | |
| 3 | >ALX30160.1 hemagglutinin [Influenza A virus | Amino acid, native sequence |
| | (A/swine/Italy/3033-1/2015(H1N2))] EUH1-17 | |
| 4 | >KR700530.1 Influenza A virus | Nucleotide, native sequence |
| | (A/swine/Italy/28762-3/2013(H1N1)) segment 4 | |
| | hemagglutinin (HA) gene, complete cds | |
| 5 | >EUH1-3 Swine optimized sequence for EUSIV- | Nucleotide, Codon optimized |
| | T8-RP production | |
| 6 | >AKJ81667.1 hemagglutinin [Influenza A virus | Amino acid, native sequence |
| | (A/swine/Italy/28762-3/2013(H1N1))] EUH1-3 | |
| 7 | >KU323308.1 Influenza A virus | Nucleotide, native sequence |
| | (A/swine/Italy/240849/2015(H3N2)) segment 4 | |
| | hemagglutinin (HA) gene, complete cds | |
| 8 | >EUH3-4 Swine optimized sequence and used in | Nucleotide, Codon optimized |
| | EUSIV-K | |
| 9 | >ALX30415.1 hemagglutinin [Influenza A virus | Amino acid, native sequence |
| | (A/swine/Italy/240849/2015(H3N2))] EUH3-4 | |
| 10 | >CY116127.1 Influenza A virus | Nucleotide, native sequence |
| | (A/swine/England/373/2010(H1N1)) hemagglutinin | |
| | (HA) gene, complete cds | |
| 11 | >EUH1-11 Swine optimized sequence for EUSIV-K | Nucleotide, Codon optimized |
| 12 | >AFR76205.1 hemagglutinin [Influenza A virus | Amino acid, native sequence |
| | (A/swine/England/373/2010(H1N1) EUH1-11 | |
| 13 | >KR700793.1 Influenza A virus | Nucleotide, native sequence |
| | (A/swine/England/61470/2013(H1N2)) segment 6 | |
| | neuraminidase (NA) gene, complete cds | |
| 14 | >EUN2-6 Swine optimized sequence for EUSIV-R | Nucleotide, Codon optimized |
| 15 | >AKJ82042.1 neuraminidase [Influenza A virus | Amino acid, native sequence |
| | (A/swine/England/61470/2013(H1N2))] EUN2-6 | |
| 16 | >KU323318.1 Influenza A virus | Nucleotide, native sequence |
| | (A/swine/Italy/248147-8/2015(H3N2)) segment 6 | |
| | neuraminidase (NA) gene, complete cds | |
| 17 | >EUN2-7 Swine optimized sequence for EUSIV-R | Nucleotide, Codon optimized |
| 18 | >ALX30429.1 neuraminidase [Influenza A virus | Amino acid, native sequence |
| | (A/swine/Italy/248147-8/2015(H3N2))] EUN2-7 | |
| 19 | >KU323247.1 Influenza A virus | Nucleotide, native sequence |
| | (A/swine/Italy/179057/2015(H1N1)) segment 6 | |
| | neuraminidase (NA) gene, complete cds | |
| 20 | >EUN1-4 Swine optimized sequence for EUSIV-R | Nucleotide, Codon optimized |
| 21 | >ALX30323.1 neuraminidase [Influenza A virus | Amino acid, native sequence |
| | (A/swine/Italy/179057/2015(H1N1))] EUN1-4 | |
| 22 | >KR700532.1 Influenza A virus | Nucleotide, native sequence |
| | (A/swine/Italy/28762-3/2013(H1N1)) segment 6 | |
| | neuraminidase (NA) gene, complete cds | |
| 23 | >EUN1-2 Swine optimized sequence for EUSIV-R | Nucleotide, Codon optimized |
| 24 | >AKJ81669.1 neuraminidase [Influenza A virus | Amino acid, native sequence |
| | (A/swine/Italy/28762-3/2013(HIN1))] EUN1-2 | |

### EXAMPLE 6: Evaluation of Vaccine Efficacy of a Multivalent IAV-S Vaccine

A study was undertaken to determine the immunogenicity and efficacy of a multivalent IAV-S vaccine comprising two-dual-HA RPs (EUSIV-T8 RP encoding EUHA1-17 & EUHA1-3 antigens and EUSIV-K RP encoding EUH3-4 & EUH1-11 antigens, Tables 1a & 2) and one-triple-NA construct (EUSIV-R encoding EUN2-6, EUN1-2 & EUN2-5 antigens, Tables 1b & 2). The adjuvanted vaccine was administered to 5 pigs in two-intramuscular (IM) vaccinations at 5 and 8 weeks of age (2 mL per dose; 3×5×10⁶ RP/dose, Vaccinates). Equal number of non-vaccinates received adjuvanted phosphate buffered saline. Immunogenicity of the vaccine was measured by quantifying HI and NI titers in sera samples collected prior to experimental infection at 10 weeks of age. The efficacy of the vaccine was tested against Gent/84 [A/swine/Belgium/113/2013 (H3N2)] challenge infection via intratracheal route at 10 weeks of age (study day 32). Vaccine efficacy against IAV-S infection induced fever i.e., raise in rectal temperature and lung lesions at 3 days post infection were measured.

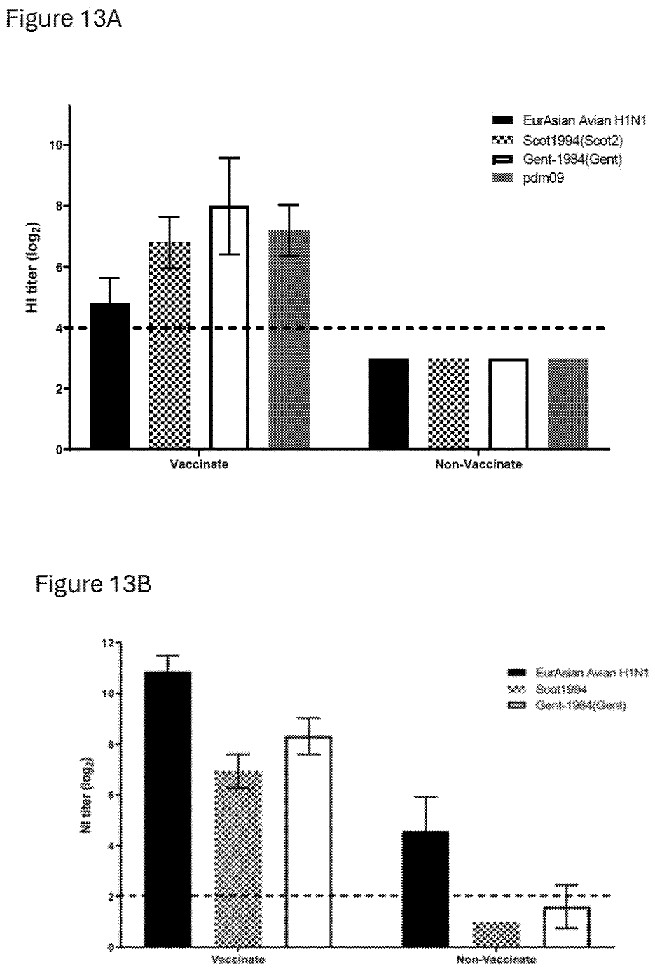

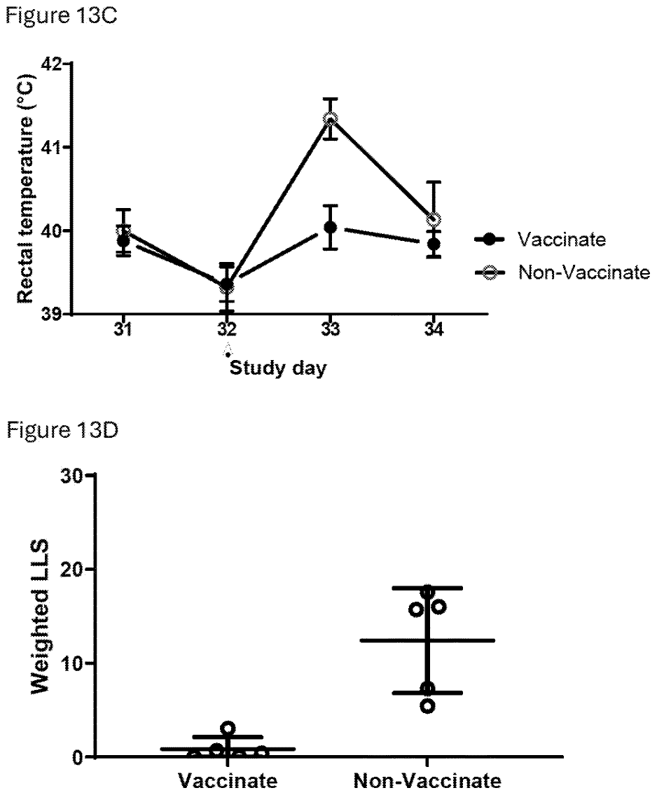

The results of this experiment are shown in FIGS. 13A, 13B, 13C and 13D. Multivalent IAV-S vaccine induced functional HI titers against heterologous IAV-S strains belonging to all four lineages (FIG. 13A) and NI titers against homologous NA antigen of all three lineages (FIG. 13B). Furthermore, multivalent IAV-S vaccine protected pigs from the experimental infection induced raise in rectal temperature, fever (FIG. 13C) and the lesions in pigs (FIG. 13D). These results demonstrate that the tested multivalent IAV-S was both immunogenic and efficacious.

### EXAMPLE 7: Evaluation of Vaccine Efficacy after ID Administration

A study was undertaken to determine the serological efficacy of a multivalent IAV-S vaccine comprising two-dual-HA RPs (EUSIV-T8 RP encoding EUHA1-17 & EUHA1-3 antigens and EUSIV-K RP encoding EUH3-4 & EUH1-11 antigens, Tables 1a & 2) and one-triple-NA construct (EUSIV-R encoding EUN2-6, EUN1-2 & EUN2-5 antigens, Tables 1b & 2). The adjuvanted vaccine was administered to 3 pigs in two-intradermal (ID) vaccinations using IDAL® needle free injector at 5 and 8 weeks of age (200 μL per dose; 3×3×10⁶ RP/dose, Vaccinates). Equal number of non-vaccinates received adjuvanted phosphate buffered saline. Immunogenicity of the vaccine was measured by quantifying HI and NI titers in sera samples collected at 10 weeks of age.

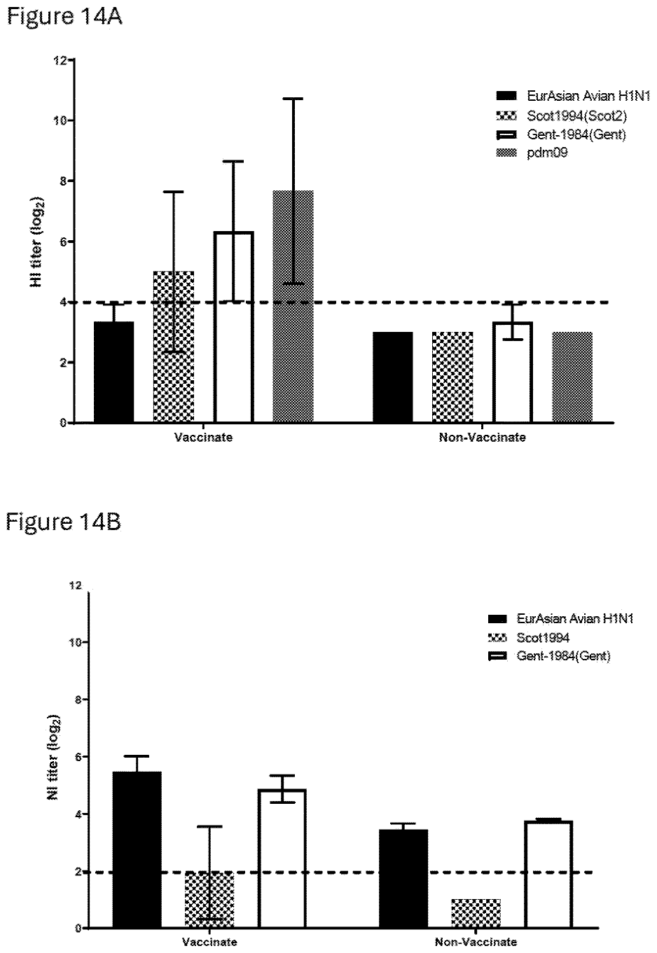

The results of this experiment are shown in FIGS. 14A and 14B. Multivalent IAV-S vaccine induced functional HI titers against heterologous IAV-S strains belonging to three out of four lineages tested (FIG. 14A) and NI titers against two out of three homologous NA antigens tested (FIG. 14B). These results demonstrate that the intradermal application of multivalent IAV-S vaccine also efficacious.

1.-24. (canceled)

25. An immunogenic composition, comprising an RNA replicon particle, wherein the RNA replicon particle comprises a nucleic acid construct comprising, in the order from 5′ to 3′ of its nucleic acid sequence:
a first nucleic acid sequence encoding a first HA antigen of IAV-S of the A/swine/Gent/1/1984-like H3N2 (Gent/84) lineage, and
a second nucleic acid sequence encoding a second HA antigen of IAV-S of the A (H1N1) pdm09 (pdm09) lineage.

26. The immunogenic composition of claim 25, wherein the RNA replicon particle is alphavirus RNA replicon particle.

27. The immunogenic composition of claim 26, wherein the alphavirus RNA replicon particle is a Venezuelan Equine Encephalitis (VEE) alphavirus RNA replicon particle.

28. The immunogenic composition of claim 25, wherein the first HA antigen is from strain A/swine/Italy/240849/2015 (H3N2).

29. The immunogenic composition of claim 25, wherein the first HA antigen encoded by the first nucleic acid sequence comprises an amino acid sequence of SEQ ID NO: 9 or an amino acid sequence having at least 90% sequence identity thereof.

30. The immunogenic composition of claim 25, wherein the second HA antigen is from strain A/swine/England/373/2010 (H1N1).

31. The immunogenic composition of claim 25, wherein the second HA antigen encoded by the second nucleic acid sequence comprises an amino acid sequence of SEQ ID NO: 12 or an amino acid sequence having at least 95% sequence identity thereof.

32. A vaccine comprising the immunogenic composition of claim 25.

33. The vaccine of claim 32, which is a nonadjuvanted vaccine.

34. The vaccine of claim 32, which comprises an adjuvant selected from the group consisting of a biodegradable oil, an oil-in-water emulsion with 2.5-50% (v/v) mineral oil, and a biodegradable oil mixed with an oil-in-water emulsion with 2.5-50% (v/v) mineral oil.

35. A method of immunizing a porcine against a swine influenza A virus, the method comprising administering to the porcine an immunologically effective amount of the vaccine of claim 32.
